(19) United States
(12) Patent Application Publication
(10) Pub. No.: US 2014/0276014 A1
(43) Pub. Date: Sep. 18, 2014

(54) SUPPORTS FOR OPTICAL SENSORS AND RELATED APPARATUS AND METHODS

(71) Applicant: Cephalogics, LLC, Boston, MA (US)

(72) Inventors: Azadeh Khanicheh, Wakefield, MA (US); Judy Walish, Brighton, MA (US); Melissa Rosen, Salem, MA (US); Michel Bruehwiler, Newton, MA (US); Jeffery J. Caputo, Winchester, MA (US); Russell L. Herrig, Canton, MA (US)

(73) Assignee: Cephalogics, LLC, Boston, MA (US)

(21) Appl. No.: 14/205,639

(22) Filed: Mar. 12, 2014

Related U.S. Application Data
(60) Provisional application No. 61/779,691, filed on Mar. 13, 2013.
(60) Provisional application No. 61/779,831, filed on Mar. 13, 2013.
(60) Provisional application No. 61/779,421, filed on Mar. 13, 2013.
(60) Provisional application No. 61/779,928, filed on Mar. 13, 2013.
(60) Provisional application No. 61/780,046, filed on Mar. 13, 2013.
(60) Provisional application No. 61/780,535, filed on Mar. 13, 2013.
(60) Provisional application No. 61/780,595, filed on Mar. 13, 2013.

(51) Int. Cl. A61B 5/00

(52) U.S. Cl. CPC A61B 5/0066; A61B 5/6803
USPC 600/425

(57) ABSTRACT

Supports for optical sensors are described. The supports may allow for placement of the optical sensors in close proximity or contact with a subject, such as supporting the optical sensors against a subject's head. The supports may include multiple pieces that can be connected or disconnected. Mechanisms for adjusting the pressure of the optical sensors against the subject may be included with the supports.

### CROSS-REFERENCE TO RELATED APPLICATIONS

This application claims priority under 35 U.S.C. §119(e) to U.S. Provisional Patent Application Ser. No. 61/779,691, entitled “OPTICAL TOMOGRAPHY SENSOR AND RELATED APPARATUS AND METHODS” filed on Mar. 13, 2013 under Attorney Docket No. C1369.70000US00, which is herein incorporated by reference in its entirety.

This application claims priority under 35 U.S.C. §119(e) to U.S. Provisional Patent Application Ser. No. 61/779,831, entitled “OPTICAL COMPONENTS FOR OPTICAL TOMOGRAPHY SYSTEMS AND RELATED APPARATUS AND METHODS” filed on Mar. 13, 2013 under Attorney Docket No. C1369.70001US00, which is herein incorporated by reference in its entirety.

This application claims priority under 35 U.S.C. §119(e) to U.S. Provisional Patent Application Ser. No. 61/779,421, entitled “DIFFUSE OPTICAL TOMOGRAPHY SYSTEMS AND RELATED APPARATUS AND METHODS” filed on Mar. 13, 2013 under Attorney Docket No. C1369.70002US00, which is herein incorporated by reference in its entirety.

This application claims priority under 35 U.S.C. §119(e) to U.S. Provisional Patent Application Ser. No. 61/779,928, entitled “SUPPORTS FOR OPTICAL SENSORS AND RELATED APPARATUS AND METHODS” filed on Mar. 13, 2013 under Attorney Docket No. C1369.70003US00, which is herein incorporated by reference in its entirety.

This application claims priority under 35 U.S.C. §119(e) to U.S. Provisional Patent Application Ser. No. 61/780,046, entitled “LINERS FOR OPTICAL TOMOGRAPHY SENSORS AND RELATED APPARATUS AND METHODS” filed on Mar. 13, 2013 under Attorney Docket No. C1369.70004US00, which is herein incorporated by reference in its entirety.

This application claims priority under 35 U.S.C. §119(e) to U.S. Provisional Patent Application Ser. No. 61/780,535, entitled “OPTICAL TOMOGRAPHY SENSOR AND RELATED APPARATUS AND METHODS” filed on Mar. 13, 2013 under Attorney Docket No. C1369.70005US00, which is herein incorporated by reference in its entirety.

This application claims priority under 35 U.S.C. §119(e) to U.S. Provisional Patent Application Ser. No. 61/780,595, entitled “OPTICAL TOMOGRAPHY SENSOR AND RELATED APPARATUS AND METHODS” filed on Mar. 13, 2013 under Attorney Docket No. C1369.70006US00, which is herein incorporated by reference in its entirety.

### BACKGROUND

1. Field

The present application relates to supports for optical sensors and related apparatus and methods.

2. Related Art

Diagnostic instruments for monitoring properties of the brain include magnetic resonance imaging (MRI) devices, computed tomography (CT) devices, microdialysis devices, transcranial Doppler devices, oxygen catheters, x-ray devices, electroencephalography devices, positron emission tomography devices, single-photon emission computed tomography (SPECT) devices, magnetoencephalography devices, ultrasound devices, and optically-based instrumentation. Some such instruments are placed in proximity to the patient's head. Optically-based sensors for analyzing medical patients are known and optical tomography is a known technique for optically inspecting a specimen.

### BRIEF SUMMARY

According to an aspect of the technology, a support as may be used for holding an optical sensor is provided. The support, in some embodiments, comprises first and second segments, the first segment being configured to couple to a rear portion of a subject's head and the second segment being in the shape of an elongated strip and configured to wrap substantially about a front portion and side portions of the subject's head. The first segment and second segment each include at least one coupler configured to detachably couple an optical sensor to an inner surface of the first and second segments, respectively. The support further comprises at least one fastener configured to detachably couple the first and second segments substantially in a loop such that the inner surface of the first segment and the inner surface of the second segment are directed inwardly toward a center of the loop. The support further comprises a first tensioner anchored on the second segment and configured to adjust a sizing of the loop and comprising at least one first strap, and a second tensioner anchored on the second segment and configured to adjust the sizing of the loop and comprising at least one second strap.

According to an aspect of the technology, a support is provided, comprising first and second segments, the first segment being configured to couple to a rear portion of a subject's head and the second segment being configured to couple to a front portion and side portions of the subject's head. The support further comprises at least one fastener configured to couple the first and second segments substantially in a loop, and at least one tensioner configured to adjust a sizing of the loop. The support may be configured to hold an optical sensor, for example in proximity to a subject's head.

### BRIEF DESCRIPTION OF DRAWINGS

Various aspects and embodiments of the application will be described with reference to the following figures. It should be appreciated that the figures are not necessarily drawn to scale. Items appearing in multiple figures are indicated by the same reference number in all the figures in which they appear.

FIG. 1 illustrates a system for performing optical tomography measurements on a subject's head, according to a non-limiting embodiment.

FIGS. 2A and 2B illustrate a top view and bottom view, respectively, of an optical sensor which may be used in the system of FIG. 1, according to a non-limiting embodiment.

FIG. 3A illustrates a top view of a subject's head against which three optical sensors according to an aspect of the present application are placed.

FIG. 3B illustrates a close-up view of a portion of FIG. 3A.

FIG. 3C illustrates an alternative configuration to that of FIG. 3A in which one optical sensor is centered on a subject's forehead and two optical sensors are positioned proximate the sides of the subject's head.

FIG. 4 illustrates in schematic form the layout of the optical sources and optical detectors of the optical sensor of FIGS. 2A and 2B, according to a non-limiting embodiment.

FIG. 5 illustrates in schematic form an example of circuitry which may be included with the optical sensor of FIGS. 2A and 2B, according to a non-limiting embodiment.

FIG. 6 illustrates an example of the circuitry in a system of the type illustrated in FIG. 1, according to a non-limiting embodiment.

FIG. 7 illustrates a detailed implementation of the circuitry in the optical sensor of FIG. 5 and host module 106 of FIG. 1, according to a non-limiting embodiment.

FIG. 8 illustrates an example of the interconnection between selected components of an optical sensor, according to a non-limiting embodiment.

FIG. 9 illustrates an example of the timing of operation of an optical sensor, according to a non-limiting embodiment.

FIG. 10 illustrates a top view of an example of the circuitry in the optical sensor illustrated in FIGS. 2A and 2B, according to a non-limiting embodiment.

FIG. 11 illustrates an example of a printed circuit board which may be used to support the circuitry of FIG. 10, according to a non-limiting embodiment.

FIGS. 12A and 12B illustrate a top view and bottom view, respectively, of a curved optical sensor which may be used in the system of FIG. 1, according to a non-limiting embodiment.

FIGS. 13A-13D illustrate multiple views of a hand-held device for holding an optical sensor according to a non-limiting embodiment.

FIG. 14 illustrates a perspective view of an alternative implementation of a hand-held device for holding an optical sensor, according to a non-limiting embodiment.

FIG. 15A illustrates a perspective view of an optical component which may be used as an optical source or optical detector in an optical sensor, according to a non-limiting embodiment.

FIG. 15B illustrates a cross-sectional view of the optical component of FIG. 15A.

FIG. 15C illustrates a perspective view of an alternative to that of FIG. 15A in terms of connecting an optical component to a support.

FIG. 15D illustrates a cross-sectional view of FIG. 15C.

FIG. 16A illustrates an exploded view of an optical source which may be used in an optical sensor, according to a non-limiting embodiment.

FIG. 16B illustrates a perspective view of the assembled version of the optical source of FIG. 16A absent the optically transparent cover 1508 of FIG. 16A.

FIG. 16C illustrates a cross-sectional view of the optical source of FIG. 16A in assembled form.

FIG. 17A illustrates an exploded view of an optical detector which may be used in an optical sensor, according to a non-limiting embodiment.

FIG. 17B illustrates a perspective view of the assembled version of the optical detector of FIG. 17A absent the optically transparent cover 1508 of FIG. 17A.

FIG. 17C illustrates a connection footprint of the optical detector of FIG. 17A.

FIG. 17D illustrates a cross-sectional view of the optical detector of FIG. 17A in assembled form.

FIG. 18 illustrates a cross-sectional view of an optical component having a tapered shape.

FIG. 19 illustrates a cross-sectional view of an alternative optical component having a flat tip.

FIGS. 20A-20C illustrate multiple views of a support engaged with a subject's head, according to a non-limiting embodiment.

FIGS. 21A and 21B illustrate different sides of a support segment of the type that may be used to engage a back portion of a head, according to a non-limiting embodiment.

FIG. 22 illustrates a support segment that may be used to engage the front and sides of a subject's head, according to a non-limiting embodiment.

FIGS. 23A and 23B illustrate different sides of a non-limiting implementation of the support segment of FIG. 22, according to a non-limiting embodiment.

FIG. 24 illustrates a piece of the support segment of FIG. 23A, according to a non-limiting embodiment.

FIG. 25 illustrates a ring which may be used to couple two pieces of a support segment, according to a non-limiting embodiment.

FIG. 26 illustrates an example of the interconnection of the support segments of FIGS. 21A and 23A, according to a non-limiting embodiment.

FIG. 27 illustrates a multi-segment support in place on a subject's head, according to a non-limiting embodiment.

FIG. 28 is a rear perspective view of the support segments of FIGS. 21A and 23A when coupled together on a subject's head, according to a non-limiting embodiment.

FIG. 29 illustrates a front perspective view of the support of FIG. 28, according to a non-limiting embodiment.

FIGS. 30A and 30B illustrate perspective views of alternative embodiments of a liner for an optical sensor of the type illustrated in FIG. 2A, according to non-limiting embodiments.

FIG. 30C illustrates a side view of a non-limiting embodiment of a portion of a liner for an optical sensor of the type illustrated in FIG. 2A.

FIG. 31A illustrates a non-limiting example of the optical sensor of FIG. 2A with a liner in place, and FIG. 31B illustrates a close-up view of a portion of the structure of FIG. 31A.

FIGS. 32A and 32B illustrate a top view and bottom view, respectively, of a device which may be used for applying a liner of the types described herein to an optical sensor, according to a non-limiting embodiment.

FIG. 33 illustrates a liner of the types described herein engaged with a device of the type illustrated in FIG. 32A, according to a non-limiting embodiment.

FIGS. 34A and 34B illustrate a manner of using a device of the type illustrated in FIGS. 32A and 32B to apply a liner of the types described herein to an optical sensor of the type illustrated in FIG. 2A, according to a non-limiting embodiment.

FIG. 35 illustrates a structure which may be used in connection an optical sensor to control how the optical sensor contact a subject, according to a non-limiting embodiment.

FIG. 36 illustrates a cross-sectional view of a structure in place on an optical sensor for controlling how the optical sensor contacts a subject, according to a non-limiting embodiment.

### DETAILED DESCRIPTION

Aspects of the present application relate to systems and methods for using optical tomography to provide and/or evaluate a condition or characteristic of a subject of interest, such as the brain of a human patient. Such evaluation may be desirable in various circumstances, such as when dealing with medical patients (which represent one example of a subject) who have suffered brain trauma, who suffer from a neurological disease (e.g., stroke), or for whom it is otherwise desirable to monitor the condition of the brain, as non-limiting examples. In some such circumstances, evaluation of the subject's condition may not be easily achieved due to physical constraints, such as the physical placement of the subject, the physical condition of the subject (e.g., open wounds, etc.), positioning of various medical equipment relative to the subject (e.g., surgical tools, other monitoring equipment, etc.), and/or obstacles in the form of hair or other objects on the target area of interest of the subject (e.g., the subject's head), among others. In some embodiments, the subject may not be able to be moved to a room having an MRI, CT scanner, x-ray machine, or other diagnostic instrument because, for example, the subject (e.g., a medical patient) may rely on life supporting systems which are incompatible with such imaging devices. Moreover, in some such circumstances, the subject may be unable to tolerate physically invasive evaluation tools or movement of the head. Such circumstances can arise, for example, in the context of neurocritical care environments. Further still, in some such circumstances long term monitoring of the subject may be desirable compared to diagnostic tools which typically provide information about only a short time (e.g., a point in time).

Accordingly, aspects of the present application provide systems for performing minimally- or non-invasive diffuse optical tomography (DOT) measurements of a subject suitable for providing information regarding one or more physical conditions or characteristics of a target portion of the subject (e.g., the subject's brain including the surface thereof, limb, torso, skin flap, organ, breast, tissue exposed by surgery, or other region of interest). Additionally or alternatively, the systems may be used to analyze such information, for example to assess a condition or characteristic of the subject (e.g., to assess a condition of the subject's brain, to assess a transplanted limb or organ, etc.). In some such embodiments, the monitoring may be performed bedside in a medical facility.

In some embodiments, the systems include a sensor (e.g., an optical sensor) configured to be placed on a subject's head while being minimally obtrusive. Optical data may be collected regarding (and in some embodiments, representing) multiple regions of the subject's brain, and in some embodiments may be collected on a continuous (or substantially continuous) basis. The data may be indicative of one or more physical conditions and/or may be suitably processed to allow for analysis (e.g., visual display) of one or more physical (e.g., biological) conditions of interest, such as oxygenated hemoglobin (HbO2) and de-oxygenated hemoglobin (HbR) levels, total hemoglobin levels (tHb), or other metrics of interest. In some embodiments, a map of tissue oxygen saturation (StO2) levels in the brain, in muscular tissue, or in any other target area of interest, may be generated. The systems may thus facilitate analysis of a subject's brain, particularly in neurocritical care environments, among others.

In those embodiments in which oxygenated, de-oxygenated, and/or total hemoglobin levels are determined, such determination may be made in any suitable manner. For example, in biological tissue, absorption of light at wavelengths in the 600 to 900 nm range depends primarily on hemoglobin, lipids, melanin and water. Absorption due to oxygenated and deoxygenated hemoglobin varies with the wavelength throughout this range in consistent and predictable ways. Thus, light absorption measurements at two or more wavelengths may be used to estimate concentrations of oxygenated and de-oxygenated hemoglobin. In a particular tissue, absorption may be estimated from detected light intensity at two or more distances from a light source. From estimates of the optical absorption at two or more wavelengths, concentrations of oxygenated and de-oxygenated hemoglobin may be estimated. Total hemoglobin concentration may be calculated as a sum of the oxygenated and deoxygenated hemoglobin concentrations.

In some embodiments, systems for performing DOT analysis of a subject's head may include multiple, physically distinct components, though not all embodiments are limited in this respect. For example, a sensor may be provided on the subject's head and a support may be provided for holding the sensor to the subject. In some embodiments, the support may hold or position the sensor relative to a subject, and thus in some embodiments the support may be considered a holder or positioner. In those embodiments in which the support holds the sensor to a subject's head, the support may be referred to as a “headpiece.” In some scenarios, more than one sensor may be provided, for example to measure and compare biological conditions of different regions/areas of interest. One or more control components for controlling the sensor may be provided remotely from the sensor. A non-limiting example of such a system according to an aspect of the present application is shown in FIG. 1. The subject may be a medical patient (e.g., a surgical patient, a patient having suffered a stroke or other brain trauma, etc.). However, various aspects described herein are not limited to use with medical patients, but rather are more generally applicable to study of various subjects for which optical tomography may provide information of interest relating to the subject.

System 100 includes a support 102, one or more sensors 104 (two of which are shown), a host module 106 (which may also be referred to herein simply as a “host”), and a central unit 108 (which may also be referred to herein as a “master”). The support 102 may support the sensor(s) 104 in relation to the head 110 of a subject (e.g., a medical patient). Thus, the support 102 may represent a headpiece in some embodiments. The system may irradiate the subject's head with optical emissions from the sensor 104 and detect and process optical emissions received from the head, including the original optical emissions emitted by the sensor 104 and/or optical emissions triggered inside the subject in response to original optical emissions from the sensor 104. The host module 106 and central unit 108 may perform various functions, including controlling operation of the sensor 104 and processing the collected data.

The system 100 may be used to provide and/or analyze information relating to various physical conditions or characteristics. For example, the intensity, phase, and/or frequency of optical signals detected by an optical detector may be used to provide information relating to various physical conditions or characteristics. In some embodiments, the system 100 may be used to provide and/or analyze information relating to absorption (within a given spectral range) of endogenous biological chromophores, such as: oxygenated hemoglobin; de-oxygenated hemoglobin; lipids; water; myoglobin; bilirubin; and/or cytochrome C oxidase. In some embodiments, the system may monitor oxygenated and de-oxygenated hemoglobin concentrations in tissue, and absorption by the other listed chromophores may be considered in determining the oxygenated and de-oxygenated hemoglobin absorptions.

In some embodiments, the system 100 may measure absorption by exogenous chromophores, such as indocyanine green (ICG) or other biologically compatible near infrared (NIR) absorbing dyes or optical tracers, which may be introduced to the subject (e.g., human tissue) in any suitable manner.

In some embodiments, alternatively or in addition to measuring absorption properties, the system 100 may measure scattering properties of a subject, such as scattering properties of biological tissue. Measured absorption properties and scattering properties may allow for determination of oxygenated hemoglobin concentration and deoxygenated hemoglobin concentration, from which one may calculate total hemoglobin concentration and tissue oxygen saturation (HbO2)/(tHb)).

In some embodiments, the system 100 may be used to determine (or partially measure) physiological indicators (or measurable quantities leading to determination of such indicators) including arterial and venous oxygen saturation, oxygen extraction fraction, cerebral blood flow, cerebral metabolic rate of oxygen, and/or regional cerebral blood flow, among others.

In some embodiments, the system may be configured to measure any of the previously described indicators or characteristics spatially. Thus, one or more images may be generated from the resulting data. In some embodiments, multiple areas or regions of a subject may be imaged substantially simultaneously (which includes simultaneous imaging), thus allowing comparison of image results for the different areas or regions.

The system 100 may have dynamic measurement properties that provide sufficient (in the physiological realm) time resolution to resolve functional (stimulus-response) activation as well as track optical tracer concentration changes. The system may be suitable for long-term real-time measurements of changes in optical absorption allowing for continuous subject monitoring (e.g., continuous monitoring of a medical patient) over extended periods and allowing for the measurement and tracking of treatment response.

The support 102, sensor 104 (which in some embodiments may be referred to as a sensor array), host module 106 and central unit 108 of system 100 may take various forms, non-limiting examples of which are described further below. The sensor 104 may be an optical sensor (generating and/or receiving optical signals) and may include suitable components for performing DOT measurements (using near infrared spectroscopy (NIRS) techniques, for example), including one or more optical sources and/or one or more optical detectors. As shown, the sensor 104 may be configured to optically couple to a subject's head (or other region of interest of a subject). In some embodiments, the sensor 104 may be flexible to conform to the subject's head.

The support 102 may hold or otherwise support the sensor 104 against the subject's head, and may have any suitable construction for doing so. In some embodiments, the support 102 may be formed of a flexible material to allow it to conform to the subject's head and/or to the sensor 104. As shown, in some embodiments the support 102 may be configured to minimize coverage of the subject, thus allowing (unimpeded) physical access to the subject over as large an area as possible. For example, as shown in FIG. 1, the support 102 may have an open-top construction such that the top of the subject's head may be accessible when the support 102 is in position. Other constructions are also possible.

Moreover, a support need not be used in all embodiments. For example, a sensor 104 may be held in a desired relation relative to a subject using a hand-held device (e.g., a handle coupled to the sensor 104). In such embodiments, the hand-held device may take any suitable form. Non-limiting examples are illustrated and described below in connection with FIGS. 13A-13D and 14.

The host module 106 may be coupled to the sensor 104 by a cabled or wireless connector 114 and may perform various functions with respect to the sensor 104, including controlling operation of the sensor 104 to at least some extent. For example, the host module may communicate control signals to the sensor 104 to control activation of the sensor 104 and/or may receive signals from the sensor 104 representative of the optical signals detected by the sensor 104. The host module 106 may also serve as a communication relay between the sensor 104 and the central unit 108, for example in some embodiments integrating or grouping data (e.g., data packets) from multiple sensors 104 into a frame prior to sending to the central unit 108. The host module may be implemented in any suitable form.

The central unit 108, which may be implemented in any suitable form, may be coupled to the host module by a cabled or wireless connection 116 and may perform various control functionality for the system. For example, the central unit 108 may include a user interface via which a user (e.g., a doctor, clinician, or other user) may select the conditions of a test or monitoring event to be performed on the subject. The central unit 108 may provide to the host module 106 suitable control signals relating to the selected test or monitoring event. The host module 106 may, in turn, provide suitable control signals to the sensor 104 to cause production and collection of optical emissions. Collected signals may then be provided to the central unit 108 via the host module 106, and the central unit may, for example, perform post processing on the signals. In some embodiments, the central unit 108 may control display of collected information, for example in textual and/or graphical form on a display 112.

While the system 100 of FIG. 1 is shown as including a distinct host module 106 and central unit 108, it should be appreciated that not all embodiments are limited in this respect. For example, in some embodiments, the host module 106 and the central unit 108 may be integrated as a single unit.

In some embodiments, an optical system such as system 100 may be used in connection with other sensing modalities. For example, the optical system may be used in combination with electroencephalography (EEG). Such a combination may facilitate, for example, monitoring of brain electrical activity as well as tissue perfusion. Thus, the system 100 is not limited to being used on its own.

According to an aspect of the application, an optical sensor is provided that includes a plurality of optical sources and a plurality of optical detectors. The optical sources and optical detectors may be formed on or otherwise connected by a common substrate, which may be flexible in some embodiments, allowing the optical sensor to be placed in contact with, and to conform to, a subject of interest or portion thereof (e.g., a subject's head). The optical sensor may also include analog and/or digital circuitry (e.g., control circuitry) for controlling collection of data by the optical sensor. The optical sensor may communicate digitally (e.g., via a digital cabled connection) to one or more remote components for receiving control signals and providing collected data to the remote components.

According to an aspect of the application, an optical structure includes a plurality of optical sources disposed on flexible circuit board strips and a plurality of optical detectors disposed on flexible circuit board strips. The flexible circuit board strips may be positioned relative to each other such that the optical sources and optical detectors collectively form an optical array. For example, the flexible circuit board strips may be interspersed or interleaved with each other. Circuitry, including analog and/or digital circuitry may also be disposed on flexible circuit board strips coupled to the flexible circuit board strips on which the optical sources and optical detectors are disposed. The entire structure may be, in some embodiments, partially or completed encapsulated in a supporting structure, such as in a flexible rubber material.

According to an aspect of the application, an optical apparatus includes an array of optical sources and optical detectors provided on a common substrate configured to contact (or otherwise be disposed in proximity to) a subject, such as a patient. The optical sources and/or optical detectors may be close to the surface of the subject, which may serve to minimize loss of light intensity as optical signals pass from the optical sources through the subject to the optical detectors. For example, in some embodiments an optical source may be positioned such that it has an emission point located within approximately 10 mm of an outer surface of the optical apparatus, within approximately 3 mm of an outer surface of the optical apparatus arranged for positioning adjacent the subject's surface, within 2 mm of the outer surface, within 1 mm of the outer surface, or any other suitable distance from the outer surface. In some embodiments an optical detector may have a detection point disposed within approximately 10 mm of the outer surface of the optical apparatus, within 3 mm of the outer surface of the optical apparatus, within 2 mm of the outer surface, within 1 mm of the outer surface, or any other suitable distance from the outer surface.

According to an aspect of the application, a method of operating an optical sensor is provided. The optical sensor may include a plurality of optical sources and a plurality of optical detectors. The optical sources may be controlled to irradiate a subject (e.g., a patient) with optical signals. The optical signals may pass through the subject and be detected by the optical detectors upon exit from the subject. In some embodiments, the optical signals from the sources may enter the subject and cause an optical emission within the subject that is then detected by the detectors. The optical detectors may generate analog signals representative of the detected optical signals (whether representing the original optical signals from the optical sources after passing through the subject or optical signals triggered internally to the subject in response to the optical signals from the optical sources), and in some embodiments the analog signals may be converted to digital signals on the optical sensor. The resulting digital signals may be transmitted to a remote component for further processing.

Aspects of the application are directed to structures for optical components including optical sources and optical detectors. In some embodiments, a similar structure may be implemented for both optical sources and optical detectors, but with optical sources including a different type of optically active element than optical detectors. In some embodiments, an optical component may include a columnar structure with an upper surface on which the optically active element, be it an optical emitter or a detecting element, is disposed. The columnar structure may include a columnar printed circuit board, and may include electrical connections for connecting to the optically active element such that electrical signals can be provided to and/or received from the optically active element.

According to an aspect of the application, an optical component is provided, which may be either an optical source or an optical detector. The optical component may be configured to have an emission/detection point raised above surrounding structures, and may in some embodiments be configured to facilitate working through (or penetrating) obstacles such as hair. In some embodiments, the optical component includes a columnar printed circuit board (PCB) having an upper surface with conductive traces thereon and having a height between approximately 2 mm and approximately 20 mm (e.g., 5 mm, 10 mm, 15 mm, or any other suitable height). The upper surface may be higher than surrounding structures. An optically active element (e.g., an optical emitter, such as a light emitting diode (LED), or an optical detecting element, such as a photodetector) may be disposed on the upper surface of the columnar PCB and electrically coupled to the conductive traces of the columnar PCB.

In some embodiments, the optically active element may be covered by one or more components. For example, an optically transparent or transmissive cover may be included with the optical component to cover the optically active element. Any such cover may be transparent (or, in some embodiments, transmissive) to wavelengths emitted by or detected by the optically active element. In some embodiments, a sleeve may be provided at least partially around the columnar PCB and the optically transparent cover. The sleeve may serve one or more functions, such as being a support (e.g., to maintain relative positioning of two or more of the constituent parts of the optical component), serving as an electrical connection (e.g., a conductive pathway), and/or performing a light blocking or isolation function.

According to an aspect of the application, an optical sensor for use in an optical tomography system is provided. The optical sensor may include one or more optical components of a type described herein. In some embodiments, multiple optical components (e.g., multiple optical sources and/or multiple optical detectors) may be provided with the optical sensor, and may be arranged in an array or other suitable configuration.

As described previously, in some embodiments an optical component may be configured to penetrate (or extend through) obstacles (e.g., hair). For example, when using optical tomography sensors to evaluate a medical patient, the optical component may need to extend through hair or other obstacles to contact the patient. In some embodiments, the optical component may be sized (e.g., having a particular cross-sectional area, a particular width, etc.) to facilitate extending through such obstacles.

The aspects and embodiments described above, as well as additional aspects and embodiments, are described further below. These aspects and/or embodiments may be used individually, all together, or in any combination of two or more, as the application is not limited in this respect.

An optical system for using DOT to analyze a subject, such as system 100 of FIG. 1, may use any suitable optical sensor 104. In some embodiments, the optical sensor may have a plurality of optical sources and/or a plurality of optical detectors, which may be arranged in an array. The optical sources and optical detectors may be coupled together mechanically to facilitate positioning with respect to the subject. For instance, the optical sources and optical detectors may be disposed on or otherwise coupled to a shared substrate which may be positionable with respect to the subject. A non-limiting example is illustrated in FIGS. 2A and 2B, which show a top view and bottom view, respectively, of an optical sensor 200 which may be used in the system of FIG. 1, according to a non-limiting embodiment.

The optical sensor 200 includes a plurality of optical sources 202 (shown with dotted fill), totaling ten in all, and a plurality of optical detectors 204, totaling eighteen in all. Collectively, the optical sources 202 and optical detectors 204 form an array in the non-limiting embodiment illustrated, and thus the optical sensor 200 may alternatively be referred to herein as a sensor array. In particular, in the non-limiting example of FIG. 2A, the optical sources 202 and optical detectors 204 are arranged in alternating rows that are offset from each other. Optical sensor 200 may be configured to be placed in contact with (or at least in close proximity to) a subject (e.g., a patient), such that the optical sources 202 irradiate the subject with optical signals (e.g., near infrared (NIR) signals) and optical detectors 204 receive the optical signals from the subject, which in some embodiments occurs after they pass through the subject. A non-limiting example is described in further detail below with respect to FIG. 3A.

FIG. 3A illustrates a top view of a subject's head 110 against which three optical sensors 200 are placed. One of the optical sensors 200 is placed centrally on the back 300 of the head 110 while the other two optical sensors 200 are placed bilaterally toward the front 301 of the head (i.e., toward the forehead).

In some embodiments, the optical sensors 200 may be considered to be pads or patches to be affixed to or otherwise held in proximity to a desired area of the subject. However, not all embodiments of sensor arrays described herein are limited in this respect.

Each of the three optical sensors 200 in FIG. 3A may irradiate the head 110 with optical signals from the optical sources of the optical sensor. The optical signals may distribute within the subject, for example across a half-sphere shape or other distribution pattern. At least a percentage of the optical signals may follow an arc (or “banana” shape) (or other path, as the exact type of path is not limiting) before exiting the head 110 and being detected by one or more optical detectors of the optical sensor. For example, referring to the optical sensor 200 identified by bracket 302, an optical signal (e.g., a light ray) 304a may be directed into the subject from an optical source 202 along the path shown in the direction of the arrows. Upon exiting the head 110, the optical signal 304a may be detected by one or more optical detectors 204 of the optical sensor 200. Similar behavior may be used to generate and detect optical signals 304b and 304c. Information about the subject may be determined from the detected optical signal, for example by analyzing the amplitude, phase, and/or frequency of the optical signal upon detection and by comparing such values to the amplitude, phase, and/or frequency of the optical signal when produced by the optical source. Any suitable signal processing may be performed related to amplitude, phase, or frequency of the optical signal 304a (or other optical signals) to determine a quantity of interest. In some embodiments, processing may involve comparing (or otherwise using) detected quantities representing an optical signal from a single optical source that is detected by multiple optical detectors located at different distances from the optical source. Because the depths to which the detected optical signals travel within the subject may depend on the distance between the optical source and the optical detector, using multiple optical detectors located at different distances from the optical source may provide information about different depths within the subject, and thus allow for comparison of such information.

As can be seen in FIG. 3A, optical signals produced by an optical source 202 of one optical sensor 200 may be detected by an optical detector 204 of a different optical sensor 200, as indicated by the path of optical signal 306. In this manner, information about a greater percentage of a target area of a subject (e.g., a patient's brain) may be determined than if data collection was limited to optical signals sourced and detected by the same optical sensor.

As described already, any suitable number and configuration of optical sensors may be used. The use of three optical sensors as shown in FIG. 3A may facilitate analysis of multiple regions of a subject's brain (or, more generally, multiple regions or portions of a subject), such as both hemispheres of a subject's brain. However, one, two, three, four, five, or more optical sensors may be used to monitor one or more properties of interest of a subject's brain. Also, the optical sensors may be arranged in manners other than that shown in FIG. 3A. For example, FIG. 3C illustrates a non-limiting alternative configuration to that of FIG. 3A in which one optical sensor 200 is centered on the front 301 of the subject's head and two additional optical sensors 200 are positioned proximate the sides of the subject's head. Other configurations are also possible.

Also worth noting with respect to FIGS. 3A and 3C is that the optical sensors 200 may be constructed such that optical signals from an optical source are not detected by an optical detector unless they pass through the head 110. Such isolation of the optical sources and detectors from each other may be beneficial, for example to minimize or avoid entirely collection of data not representative of the subject. Such isolation may be achieved in multiple ways, including mechanically coupling the optical sources and optical detectors of the optical sensor to each other with an optically opaque material, as described in further detail below, and/or using light shields, shielding tubes, and/or light guides in connection with the optical sources and/or optical detectors, as non-limiting examples.

As should also be appreciated from FIGS. 3A and 3C, optical sensors according to an aspect of the present application may be placed in contact with a subject, such that the optical sources and/or optical detectors of the optical sensor may be close to the subject. FIG. 3B further illustrates the point, and provides a close-up view of the portion of FIG. 3A identified by box 310.

As shown, the optical sensor 200 includes an outer surface 312 configured to contact the subject's head 110. In the non-limiting embodiment illustrated, the outer surface 312 corresponds to the outer surfaces of optical source 202 and optical detector 204, though not all embodiments are limited in this respect. The optical source 202 includes an active emitter (e.g., an LED) having an emission point 314 (e.g., the emission point 314 may correspond to the location of the LED within the optical source 202), while the optical detector 204 (e.g., a photodetector) has a detection point 316 (e.g., the detection point 316 may correspond to the location of the photodetector within the optical detector 204). The emission point 314 and/or detection point 316 may be separated from the subject's head 110 by a distance dl. In some embodiments, dl may be small. For example, dl may be less than approximately 10 mm, less than approximately 5 mm, less than approximately 3 mm, less than approximately 2 mm, less than approximately 1 mm, or any other suitable distance. By configuring the optical sensor in some embodiments such that the distance dl is small, the light intensity lost as the optical signals pass from the optical sources into the subject and out to the optical detectors may be minimized. Furthermore, as shown, the subject may be impressed (at least slightly) by the optical source 202 and/or optical detector 204 which may improve the transmission of signals between the optical source 202 and the subject 110, and between the subject 110 and the optical detector 204. The distance dl need not be the same for the optical source 202 and the optical detector 204 in all embodiments. Rather, the emission point 314 and detection point 316 may be positioned at different distances from the outer surface 312 of the optical sensor.

FIG. 3B illustrates only a single optical source 202 and optical detector 204. However, it should be appreciated that in some embodiments a plurality (e.g., all) of the optical sources and/or optical detectors of an optical sensor may be configured with respect to the subject as shown, i.e., within the distance dl of the subject.

Moreover, it should be appreciated that while FIG. 3B illustrates a configuration in which the outer surface 312 corresponds to the surface of the transparent cover 318, described further below), not all embodiments are limited in this respect. For example, in some embodiments one or more additional layers may optionally be disposed on the transparent cover 318, with the outer surface 312 corresponding to the outermost surface of such layers.

The optical sources 202 and optical detectors 204 may have any suitable constructions. For example, each of the optical sources 202 and detectors 204 may include a transparent cover 318, for example being a lens formed of a resin or other suitable optically transparent material. In some embodiments, the transparent cover 318 may function as a light guide, and thus be alternatively referred to as a light guide (e.g., a shaped light guide), or in some embodiments a lens. Its shape may be selected to maximize the light intensity entering the subject from the optical source. The transparent cover 318 may be formed of a hard (e.g., non-compressible, such as polycarbonate) or soft (e.g., compressible, such as silicone) material. In some embodiments, a soft material may be selected to improve comfort for the subject, since the optical sources may be forced against the surface of the subject (e.g., being placed in contact with a patient's head).

The optical sources 202 may optionally include a filter 322, and the optical detectors 204 may optionally include a filter 324. Such filters may be integrated with the transparent covers 318 (e.g., being a single component). Other components may optionally be included.

Thus, in some embodiments, the only thing between the emission point 314 and the subject may be a filter and a lens/cover (e.g., transparent cover 318), and likewise the only thing between the detection point 316 and the subject may be a filter and a lens. Other constructions are possible.

FIG. 4 illustrates in schematic form the layout of the optical sources 202 and optical detectors 204 of the optical sensor 200. Again, there are ten total optical sources 202 (represented by circles in FIG. 4 and numbered 1-10 for ease of explanation) and eighteen total optical detectors 204 (represented by squares in FIG. 4 and numbered 1-18 for ease of explanation) in the non-limiting embodiment of optical sensor 200, but it should be appreciated that other numbers of optical sources and/or optical detectors may be used, such that the various aspects of the present application are not limited to using any particular number of optical sources and optical detectors in an optical sensor. For example, according to one embodiment an optical sensor may be substantially the same as optical sensor 200 but include only two optical sources and two optical detectors. Other configurations are also possible. The number of optical sources and/or optical detectors may be selected in dependence upon a desired application of the optical sensor, keeping in mind data processing goals/constraints (e.g., a larger number of optical detectors will lead to a greater amount of data to process), and the desired size of the region of the head (or other subject) to study, among other potential considerations.

As described previously, and as illustrated in FIG. 4, embodiments of the present application provide for an optical sensor for which more than one optical detector 204 detects optical signals produced by a particular optical source 202. For example, referring to FIG. 4, optical detectors 8, 9, and 15 may all detect optical signals produced by optical source 5 (as may other optical detectors). Optical detectors 8, 9, and 15 are located at increasing distances L1, L2, and L3, respectively, from the optical source 5, and may be considered as first nearest neighbor to optical source 5, second nearest neighbor to optical source 5, and third nearest neighbor to optical source 5, respectively. Higher order nearest neighbors (e.g., fourth nearest neighbor, fifth nearest neighbor, etc.) may also detect optical signals in some embodiments, depending on factors such as the strength of the optical signals produced by the optical sources, the distances between the optical sources and optical detectors, and the material into which the optical signals are being sent (e.g., tissue). In some embodiments, the optical detectors receive optical signals with power between approximately 0.01 nW and 10 μW. Non-limiting examples of values of L1, L2, and L3 are provided below.

Detection of an optical signal from an optical source with multiple optical detectors may be beneficial for providing an increased amount of data about a subject as opposed to if only a single optical detector detected the optical signals produced by a given optical source. The greater the amount of data, the more robust the analysis of the subject may be. However, greater signal processing (and therefore signal processing resources) may also be needed. As a non-limiting example, assuming that first, second, and third nearest neighbor optical detectors in FIG. 4 are configured to detect optical signals, then the illustrated configuration provides for 108 channels of information (broken down as 40 first nearest neighbor channels, 52 second nearest neighbor channels, and 16 third nearest neighbor channels).

The optical sources 202 of the optical sensor 200 may emit any suitable wavelengths of optical radiation. As previously described, in some embodiments the optical sources may operate in the infrared spectrum, and in some embodiments within the NIR (near infrared) spectrum. In some embodiments, the optical sources may operate in the visible (or a portion thereof) through NIR spectrum. In some embodiments, the optical sources may emit wavelengths in the visible spectrum. As non-limiting examples, each of the optical sources may emit wavelengths between approximately 500 nm and approximately 1,100 nm, between approximately 600 nm and approximately 1,000 nm, between approximately 650 nm and approximately 950 nm, a wavelength of approximately 650 nm, approximately 700 nm, approximately 750 nm, approximately 800 nm, approximately 850 nm, approximately 900 nm, approximately 920 nm, approximately 925 nm, approximately 950 nm, or any other suitable wavelengths.

Also, in some embodiments the optical sources of the optical sensor 200 need not all emit the same wavelengths. For example, a first optical source may emit a first wavelength (e.g., approximately 650 nm) and a second optical source may emit a second wavelength (e.g., approximately 800 nm). The use of multiple wavelengths may facilitate detection of various quantities of interest with respect to the subject, since different wavelengths of the radiation may behave differently when passing through the subject.

The optical detectors may detect the wavelengths emitted by the optical sources. In some embodiments, all the optical detectors may be capable of detecting any of the wavelengths emitted by any of the optical sources. In such embodiments, all the optical detectors may be substantially identical to each other. However, in some embodiments different optical detectors may be capable of detecting different wavelength ranges from each other.

In some embodiments, the optical sensor 200 may be used to provide information about the concentration of oxygenated or deoxygenated hemoglobin (or both) in tissue of a subject (e.g., the concentration of oxygenated and/or deoxygenated hemoglobin in a subject's brain, muscle or other tissues). Thus, the wavelengths of radiation used by the optical sensor 200 may be selected to facilitate collection of such information. In some embodiments, the wavelengths utilized by the optical sensor 200 may be approximately equally dispersed over the range from approximately 650 nm to approximately 950 nm. A broader spectrum may be used at the higher end of this range, in some embodiments. A narrower range (i.e., narrower than 650 nm to 950 nm) may be used in some embodiments, for example those embodiments in which only two to four wavelengths are to be used. In some embodiments, only two wavelengths may be used, with one below the isosbestic point of hemoglobin, which is about 800 nm, and one above (e.g., one wavelength below approximately 765 nm and one wavelength above approximately 830 nm).

As described previously, in some embodiments the optical sources and optical detectors of an optical sensor may be mechanically coupled together, for example to facilitate relative positioning and spacing of the components with respect to a subject. In the example of FIG. 2A, the optical sources 202 and optical detectors 204 are at least partially encapsulated in a support structure 206, which may be a standalone component moveable by hand or with suitable positioning tools (e.g., a handle). In some embodiments, the optical sources 202 and/or optical detectors 204 may be fully encapsulated by the support structure 206, which may form a coating layer over the optical sources and/or optical detectors. In such embodiments, the coating layer may be optically transparent.

In some embodiments, the support structure 206 may be flexible, for example, being able to flex about one or more axes (e.g., in two orthogonal directions), such as about the x- and y-axes in FIG. 2A. Such flexibility may facilitate conforming the optical sensor to a subject to achieve satisfactory optical coupling. For example, by conforming the optical sensor to the subject, a large percentage (and in some embodiments, all) of the optical sources and optical detectors may contact the subject. In those embodiments in which the support structure 206 is flexible, any suitable material may be used to form the support structure, such as silicone, urethane, or any other suitable flexible material. In some embodiments, the support structure 206 may be formed of a material having a hardness between approximately 20 A and approximately 60 A durometer, with suitable tear strength and elongation. In some embodiments, the support structure 206 may be formed of a material having an elongation of at least 150%, between approximately 100% and approximately 800%, any value within that range, or any other suitable value. In some embodiments, the support structure 206 may be formed of a medical grade resin.

In some embodiments, including some of those in which the support structure 206 is flexible, the support structure 206 may be formed of an optically opaque material to optically isolate the optical sources and detectors from each other, as previously described in connection with FIG. 3A. For example, the support structure may be formed of a black (biocompatible) rubber (e.g., a rubber including carbon black, non-latex rubber, etc.) or other suitable optically opaque material (being opaque to the wavelengths of radiation used by the optical sources). The optical sources and/or optical detectors may partially protrude from the support structure 206. For example, the optical sources may protrude from the support structure 206 by an amount sufficient to allow the optical sources to direct optical signals toward the subject. The optical detectors may protrude from the support structure 206 by an amount sufficient to allow the optical detector to receive optical signals exiting the subject.

In some embodiments, the support structure 206 may be formed of a substantially optically transparent material. In such embodiments, if it is desired to prevent optical signals from the optical sources passing through the transparent material and being detected by the optical detectors of the optical sensor, other techniques (other than using an opaque support structure 206) may be used to prevent such signal detection. For example, a liner of the types illustrated and described herein may be used, as will be described further below in connection with FIGS. 30A-30C. Additionally or alternatively, the support structure 206 may be formed of a material whose light transmissive properties are dependent on angle, and for which optical signals from an optical source of the optical sensor are incident on the support structure 206 at an angle for which the support structure 206 is not transmissive. As a further alternative, the support structure 206 may be formed of a material whose light transmissive properties are controllable (e.g., like a shutter), and suitable control may be exercised to prevent undesirable tunneling or channeling of optical signals from an optical source through the support structure 206 to an optical detector of the optical sensor 200.

In some embodiments, the support structure 206 may be formed of a material that is not electrically conductive (e.g., an electrical insulator, such as rubber or resin).

As shown in FIG. 2B, the bottom side of the support structure 206 may be substantially flat in some embodiments, and in some embodiments none of the optical sources or optical detectors may protrude from the bottom of the optical sensor 200. Rather, as in the non-limiting example of optical sensor 200, some embodiments of an optical sensor may be configured such that all the optical sources and optical detectors are disposed on the same side of the optical sensor. However, other configurations are possible.

In some embodiments, the bottom side of the support structure 206 may have one or more features to provide the support structure with increased flexibility. For example, the bottom side of the support structure 206 may include grooves, channels, dimples, indentations, or other suitable features to increase the flexibility of the support structure 206.

The optical sensor 200 may also include control circuitry (or control electronics) for controlling operation of the optical sources 202 and/or optical detectors 204, including analog and/or digital circuitry. The circuitry may take any suitable form, some examples of which are described in further detail below. When such circuitry is included, it may be positioned at any suitable location(s) with respect to the optical sensor. For example, the circuitry may be grouped into modules positioned at the periphery (e.g., along a single edge) of the optical sensor. Placement of the circuitry of the optical sensor at an edge may minimize or simplify the placement of electrical connections for communicating between the optical sensor and remote components of an optical system. As a result, access to a subject (e.g., a patient) may be maximized when the optical sensor 200 is in place. Referring to FIG. 2A, the optical sensor 200 may include a first circuitry module 208a, a second circuitry module 208b, and a third circuitry module 208c. The circuitry modules 208a-208c may be integrated circuit packages or may take other forms and, as shown, may be encapsulated (partially or fully) by the support structure 206.

Various types of circuitry may be included in connection with or as part of the optical sensor 200. The optical sources 202 and/or optical detectors 204 may be analog components and thus analog circuitry may be included with the optical sensor 200. For example, the optical sources 202 may be light emitting diodes (LEDs), and therefore it may be desirable for the optical sensor 200 to include analog drive circuitry (e.g., an LED controller) configured to control, at least in part, one or more (e.g., all) of the LEDs. For example, the drive circuitry may control the ON/OFF state of the optical sources (and therefore the duration of the optical signals emitted by the optical sources), the frequency modulation of the optical sources and/or the emission intensity and power of the optical sources (e.g., by controlling the current to the optical sources). The optical detectors may be photodetectors (e.g., photodiodes, phototransistors, or any other suitable type of photodetectors) and may be coupled to analog receive circuitry, such as an amplifier, a filter, or other signal conditioning circuitry. The analog receive circuitry may be configured to receive an analog signal from one or more (e.g., all) optical detectors of the optical sensor. In some embodiments, a microcontroller may also be provided with the optical sensor 200 and may perform any of various functions, including any one or more of controlling acquisition of optical signals by the plurality of optical detectors, performing demodulation of signals acquired from the plurality of optical detectors, and serving as a communication interface between the optical sensor 200 and a remote component, such as host module 106.

In some embodiments, both analog and digital circuitry may be included with the optical sensor 200. For example, as described above, the optical sources and/or optical detectors may be analog components and therefore it may be desirable in some embodiments to include analog drive and/or analog receive circuitry with the optical sensor 200. However, it may also be desirable to perform some digital functions, such as digital signal processing, on the optical sensor itself before sending any resulting signals off the optical sensor to a remote device. Thus, the optical sensor 200 may include, in some embodiments, an analog-to-digital converter (ADC), for example to convert analog signals received by the optical detectors 204 into digital signals. In some embodiments, the microcontroller includes the ADC.

In some embodiments, a field programmable gate array (FPGA) and/or application specific integrated circuit (ASIC) may be provided to perform one or more functions. For example, an FPGA may perform some digital functions, and in some embodiments a mixed signal FPGA may provide both digital and analog functions such as analog-to-digital conversion, digital-to-analog conversion, signal conditioning, and digital logic. In some embodiments, an ASIC may provide one or more analog and/or digital functions, such as any of those previously described.

FIG. 5 illustrates in schematic form a non-limiting example of circuitry which may be included with the optical sensor 200. For purposes of explanation, the optical sources 202 are described in the context of FIG. 5 as being LEDs and the optical detectors 204 as photodetectors, though not all embodiments are limited in this respect.

As shown, the optical sensor 500 may include an LED 502 coupled to optics 504 (e.g., a lens) to produce an optical signal to irradiate a subject 506. Receiving optics (e.g., a lens) 508 provide the optical signal to a photodetector 510. Circuitry for controlling operation of the LED 502 includes an LED controller 512, as well as the microcontroller 514. The microcontroller 514 may send digital signals to the LED controller 512, which may in turn provide an analog control signal to the LED 502. Circuitry for processing the signals received by the photodetector 510 include a transimpedance amplifier (TIA) 516 (which converts a received current to a voltage and amplifies the voltage), an ADC 518, and the microcontroller 514.

The microcontroller 514 may perform various functions, such as any of those described elsewhere in the present application as being performed by a microcontroller, or any other suitable functions. According to an embodiment, the microcontroller 514 may execute firmware suitable to perform one or more of the following functions: awaiting a “start of frame” signal from a host; switching between optical sources of the optical sensor; enabling and controlling the optical sources including performing frequency modulation; providing a sampling clock to an ADC; controlling signal acquisition by the photodetector 510 and connected receive circuitry; demodulation of acquired signals (e.g., Fast Fourier Transform (FFT) or other suitable demodulation depending on the type of modulation used for optical signals produced by the LED 502); or communication handler between the optical sensor 500 and any remote components, such as host module 106 in FIG. 1, for example by compiling and transmitting communication packets to the host module.

FIG. 6 illustrates a non-limiting example of the circuitry in a system of the type of FIG. 1. For purposes of illustration, the system 600 includes an optical sensor illustrated as being optical sensor 500 of FIG. 5, which is described in detail above. As previously described in connection with FIG. 1, the optical sensor may be coupled to a host module 106. The host module 106 may include a microcontroller 602. The host module in turn may be connected to a central unit 108, which itself may include one or more processors.

The host module 106 may be connected to the optical sensor (or multiple optical sensors) via a cabled or wireless connector 114. In some embodiments, the host module 106 may be connected to multiple optical sensors via a single cable which splits to go to each optical sensor. In some embodiments, the host module 106 may be connected to multiple optical sensors via respective cables. FIG. 6 illustrates a cabled connector 604. As described previously, the optical sensor 500 may include digital circuitry and thus communication between the optical sensor 500 and the host module 106 may occur in the digital domain. Thus, the connector 604 may be a digital connector, such as a low voltage differential signaling (LVDS) cable, a universal serial bus (USB) connector, Ethernet connector, RS-232 connector, RS-432 connector, or an RS-485 connector, among other possibilities. The host module may also have an auxiliary input port 606 (e.g., an 8-bit communication line or any other suitable communication line) to receive auxiliary input.

This auxiliary input port may be used to capture digital information from an external device and synchronize in time (to the time resolution of a frame) the auxiliary data input with the data from the optical sensor 200. In some embodiments, the data provided on the auxiliary input port 606 may be provided with each frame of data from the optical sensor 200 to the central unit 108. As a non-limiting example, the timing and type of a stimulus given to a subject may be captured, for example in the context of a brain stimulus-response study.

In some embodiments, the host module 106 may also include an auxiliary output port 610, for example being configured to output data (e.g., 8 bits of data or any other suitable amount) to provide synchronization, frame count, Host status or configuration, or optical sensor status or configuration data to an external device. For example, such data may be provided in the context of synchronous monitoring.

In some embodiments, any auxiliary input and output ports of the host module 106 may be used for functional and performance testing and verification of the host module 106. Other uses for the auxiliary input and output ports are also possible.

The host module 106 may perform any suitable functions, such as any of those previously described in connection with host module 106. For example, the microcontroller 602 may execute firmware to perform one or more of the following functions: control of frame rate timing of the optical sensor; acting as a communication relay between the optical sensor and the central unit; consolidating or integrating data from multiple optical sensors into a single data packet; outputting auxiliary data to the auxiliary output port 610, or recording auxiliary input received over the auxiliary input port 606.

The central unit 108 may be a computer (e.g., a desktop computer, laptop computer, tablet computer, etc.) or other processing unit (e.g., a personal digital assistant (PDA), smartphone, etc.) and may be configured to perform one or more functions of the types previously described, for example by execution of suitable software and/or firmware. For example, the central unit 108 may perform post processing on signals detected by the photodetector 510 (e.g., performing unit conversion of the signals into optical power), though such functions may alternatively be performed by the host module 106 in some embodiments. The central unit may control and perform display of information, in image form, graphical form, textual form, or any other suitable form. In some embodiments, the central unit 108 may include a display 112 upon which information is displayed, for example to a clinician or other user. The displayed information may be representative of physical conditions (e.g., biological conditions) or characteristics of a subject detected by the optical sensor, such as hemoglobin levels (e.g., oxygenated hemoglobin, deoxygenated hemoglobin, total hemoglobin, or tissue oxygenation saturation). In some embodiments, the central unit may control analysis and/or display of images and/or information relating to two or more regions (or portions) of a subject's brain simultaneously (e.g., two hemispheres of the subject's brain). For example, referring to FIG. 3A, an image of both hemispheres 308a and 308b of a subject's brain may be produced from information collected by the three illustrated optical sensors, and such images may be displayed to a user, for example to allow for analysis of a condition or characteristic of the subject.

As described previously, aspects of the application provide for continuous monitoring of physical characteristics and/or conditions of a subject. Thus, in those embodiments in which information is presented to a user (e.g., via a visual display), such display may be continuous, and may be updated continuously. Moreover, in some embodiments it may be desired to track, trend, and display changes of monitored conditions or characteristics of the subject, thus providing historical data for comparison. As an example, a user (e.g., a doctor) may analyze current data provided by an optical sensor as well as scrolling through previously collected data to do a comparison of how a property of interest (e.g., hemoglobin levels) has changed with time.

As described previously, the central unit 108 and host module 106 may be connected by a cabled or wireless connector 116. As a non-limiting example, the two may be connected by a TCP/IP (Ethernet) connection 608, though other connection types are also possible.

FIG. 7 illustrates a non-limiting example of a detailed implementation of the circuitry of optical sensor 500 and host module 106. As shown, the optical sensor 500 may include the microcontroller 514, the LED controller 512 coupled to the array of optical sources 202, a bank 701 of TIAs 516 coupled to the optical detectors 204, and a bank 706 of ADCs 518 arranged in a daisy chain configuration and coupled to the bank 701 of TIAs. In some embodiments, the TIAs 516 may be located as closely as possible to the optical detectors 204. The use of the described daisy chain configuration may itself minimize signal path length from the optical detectors 204 to the TIAs 516 to the ADCs 518, thus improving signal path quality and minimizing signal corruption from external sources. However, it should be appreciated that not all embodiments utilize the described daisy chain configuration. For example, in some embodiments, the components may be arranged in parallel or funnel to a single ADC.

The optical sensor 500 may also comprise an LVDS driver 702 for an LVDS connection (e.g., connector 604) between the optical sensor 500 and the host module 106, which may couple to an LVDS module 705 in the host module 106. The host module 106 may also include a power supply connector 708 coupled to a power management block 704 in the optical sensor 500 to provide power to the optical sensor 500. The power management block 704 may include one or more power modules 710a-710c to provide a desired voltage level to one or more components of the optical sensor 500, as shown (e.g., the power modules may provide respective voltage levels). The optical sensor may also include an oscillator 712 to provide a reference clock signal to the microcontroller 514.

The configuration of FIG. 7 is not limiting of the various aspects described herein. Other circuitry components and configurations may be used.

FIG. 8 provides further detail of an example of the operation of certain components of FIG. 7 to acquire optical signals. In particular, FIG. 8 illustrates a manner of operation of the signal acquisition chain comprising the optical detectors 204, ADCs 518, and microcontroller 514. In the non-limiting example illustrated, three ADCs are included, and are identified as ADCs 802a-802c. There are eighteen optical detectors 204. Each of the ADCs 802a-802c is coupled to six optical detectors 204 to receive the detected signals from the optical detectors. Each of the ADCs 802a-802c also receives a clocking signal 804 from the microcontroller 514, which may be approximately 18 MHz or any other suitable frequency.

The ADCs 802a-802c may be arranged in a daisy chain configuration as previously described in connection with FIG. 7. Thus, the signals from ADC 802c are provided to ADC 802b, and the signals from ADC 802b are provided to ADC 802a. The output of ADC 802a is coupled to an input of microcontroller 514 to provide digital data to the microcontroller 514. The microcontroller 514 may then construct communication packets to send to the host module 106.

In operation, all the optical detectors 204 may sample simultaneously. The sampling rate may be any suitable sampling rate, and in some embodiments may be between approximately 30-40 kHz, approximately 35 kHz, or any other suitable rate. In some embodiments, the wavelength of the optical sources may be isolated on the receiving side via frequency encoding techniques. For example, the optical signals from the optical sources may be frequency encoded (e.g., in the kHz range), and frequency decoding/demodulation may be performed on the receiving side.

In operation, the optical sources of an optical sensor may be cycled sequentially, a non-limiting example of such operation being illustrated in FIG. 9.

FIG. 9 provides a non-limiting example of the relative timing of operation of the optical sensor 200 according to a non-limiting actuation sequence. For purposes of explanation, the optical sources 202 are referred to as optical source 1, optical source 2, . . . , optical source 10. The optical sensor may be operated such that each of the optical sources is activated only once during a frame, and in isolation of the other optical sources. Namely, a first optical source (“optical source 1”) may be activated during a time slot 902, at which time all the optical detectors 204 are sampled. The signals from the optical detectors 204 may be provided to the microcontroller 514 in the manner previously described in connection with FIG. 8. The microcontroller 514 may demodulate the signals in turn from each of the optical detectors 204 during a time slot 904. The demodulation may involve any suitable processing depending on the type of modulation used for the optical signals generated and detected by the optical sensor.

During a time slot 906, the microcontroller may packetize and transfer data to the host module 106 representing the detected optical signals.

A buffer period 908 of relatively short duration (e.g., 1 millisecond) may then be observed to ensure no overlap in the data processing of the optical sources. Subsequently, the same sequence of events may be repeated for the second optical source (“optical source 2”), and so on for all the optical sources, as shown in FIG. 9.

Alternative manners of operation are also possible. For example, in some embodiments parallel data processing may be performed, allowing for sampling of the optical sources to be performed nearly sequentially, i.e., with little or no time between the sampling of one optical source and another. In such embodiments, demodulation of received optical signals (e.g., time slot 904) and data transfer (e.g., time slot 906) may be performed substantially in parallel with the sampling operation.

A frame is completed after all the optical sources of the optical sensor have been activated. Any suitable frame rate may be used to provide a desired rate of data collection. As previously described, the host module may in turn provide the collected data to the central unit 108, which may optionally perform further processing and which may, in some embodiments, generate and display in image form, graphical form, and/or textual form data about one or more characteristics of the subject.

As should be appreciated from the foregoing description of FIGS. 1, 2A-2B, and 5-7, aspects of the present application provide an optical sensor and optical system which do not include any fiber optics (also referred to herein as “optical fibers”). Fiber optic bundles are therefore not used to communicate optical signals to/from the optical sensor and a remote component (e.g., host module 106), but rather a digital communication line (e.g., connector 604) may be used, which may simplify construction of the system. In addition, avoiding the need for fiber optic bundles may make the system more practical to use, for example by reducing the weight of the system and the number of separate connections between the optical sensor and any remote components. Thus, patient comfort and accessibility to the patient may be increased compared to if fiber optics were used to communicate between the optical sensor and any remote components. Moreover, signal losses associated with the use of fiber optics may be avoided.

In some embodiments, an optical system may lack any fiber optics for communicating between an optical sensor of the system and a remote component (e.g., host module 106), even if one or more fiber optics may be used on the optical sensor itself to optically couple the optical sensor to the subject. Any such fiber optics on the optical sensor itself may be short, for example less than two inches in length, less than one inch in length, or any other suitable length. In such embodiments, it should be appreciated that an optical system may lack any fiber optics having a length greater than approximately two inches, which may provide one or more of the benefits described above with respect to systems lacking fiber optics between an optical sensor and a remote component.

Also, aspects of the present application provide optical systems and optical sensors which need no optical fibers to irradiate a subject with optical signals or detect optical signals from the subject. For instance, optical fibers are not needed to transmit an optical signal exiting a subject to a detector located remotely from the subject, and neither is any optical fiber needed to transmit to a subject an optical signal produced by an optical source located remotely from the subject. Rather, as described previously (e.g., in connection with FIGS. 2A and 3A-3C), an optical sensor, according to an aspect of the present application, may be configured to directly contact the subject, such that the optical sources and optical detectors may be in close proximity to the subject. Despite the system including, in some embodiments, a host module and/or central unit which may be located up to several feet or more away from the optical sensor, no optical fibers are needed. Thus, it should be appreciated that embodiments of the present application provide an optical system and/or optical sensor entirely lacking any fiber optics.

Referring again to FIGS. 2A and 2B, the optical sensor 200 may have any suitable dimensions. As previously described, in some embodiments the optical sensor 200 may be configured to contact a subject's head, for example as shown in FIGS. 1 and 3A. Furthermore, an optical system of the type illustrated in FIG. 1 may be configured to include multiple optical sensors, as previously described, for example, in connection with FIG. 3A. Thus, according to some embodiments, the optical sensor 200 may be dimensioned to conform to and contact a subject's head while leaving room for additional optical sensors to also contact the head. Furthermore, as previously described, in some embodiments it may be desirable for the optical sensor to be minimally obtrusive, for example to leave room for accessing the subject (e.g., a patient) with other instrumentation/tools.

According to a non-limiting embodiment, the optical sensor 200 may have a length (e.g., in the y-direction in FIG. 2A) of between approximately 50 and 200 mm, between approximately 75 and 150 mm, between approximately 100 and 130 mm, approximately 110 mm, approximately 120 mm, approximately 125 mm, or any other suitable length. The optical sensor may have a width (in the x-direction in FIG. 2A) of between approximately 40 and 150 mm, between approximately 50 and 125 mm, between approximately 60 and 80 mm, approximately 70 mm, approximately 75 mm, or any other suitable value. The thickness of the optical sensor 200 may be between approximately 4 and 30 mm, between approximately 5 and 15 mm, approximately 10 mm, or any other suitable value.

The optical sources 202 and optical detectors 204 of the optical sensor 200 may be spaced by any suitable distances. For example, first nearest neighbor optical detectors (those optical detectors of an optical sensor array that are most closely spaced with respect to an optical source) may be within approximately 10-20 mm of the optical source (e.g., the distance L1 shown in FIG. 4 may be between approximately 10-20 mm). Second nearest neighbor optical detectors (e.g., separated by a distance L2 from an optical source, as shown in FIG. 4) may be within approximately 20-35 mm of the optical source. Third nearest neighbor optical detectors (e.g., separated by a distance L3 from an optical source, as shown in FIG. 4) may be within approximately 35-50 mm of the optical source. Other spacing values are also possible, as those described are non-limiting examples.

The optical sources 202 and optical detectors 204 may have any suitable dimensions. As mentioned, in at least some embodiments it may be desirable to have the optical sources and optical detectors close to the subject. Accordingly, in some embodiments the optical sources and/or optical detectors may be short, for example less than approximately 10 mm in height (in the z-direction of FIG. 2A), less than approximately 6 mm in height, less than approximately 5 mm in height, or may have any other suitable height. In some embodiments, the optical sources and/or optical detectors may be configured to work through (i.e., penetrate) hair or other obstacles. For example, in the context of using the optical sensor 200 to monitor a subject's brain, the presence of hair may complicate achieving good contact between the optical sensor and the subject's head. Suitable design of the optical sources and/or optical detectors may facilitate their ability to work through the hair and therefore reach the subject's scalp. Thus, in some embodiments, the optical sources and/or optical detectors may be thin, for example having a width w (see FIG. 3B) (which may, in some embodiments be a diameter if the optical sources and/or optical detectors have a circular cross-section) less than approximately 10 mm, less than approximately 8 mm, less than approximately 7 mm, less than approximately 5 mm, less than approximately 4 mm, or any other suitable value.

In some embodiments, the optical sensor may be configured to directly contact the surface of a subject's brain, for example during brain surgery. The optical sensor may have any suitable configuration, including any suitable dimensions, for such functionality.

FIGS. 2A-2B illustrate an external view of the optical sensor. There may be further internal structure in some embodiments for providing electrical and/or mechanical interconnection of the components of the optical sensor, which may take any suitable form.

For instance, as previously described, in some embodiments it may be desirable for the optical sensor to be flexible, and thus the optical sources and optical detectors may be mechanically and/or electrically coupled via flexible internal structures. FIG. 10 illustrates a top view of a non-limiting example of such an internal structure.

The structure 1000 of FIG. 10 comprises the optical sources 202, optical detectors 204, and circuitry modules 208a-208c previously described in connection with FIGS. 2A-2B. The optical sources 202 are mechanically interconnected to each other by flexible circuit board strips 1002. In the non-limiting embodiment shown, there are five flexible circuit board strips 1002 interconnecting the ten optical sources 202 (two optical sources 202 being disposed on each of the flexible circuit board strips 1002). Similarly, the optical detectors 204 are disposed on flexible circuit board strips 1004, which may be the same type of circuit board strips as circuit board strips 1002. As can be seen, the circuit board strips 1002 and 1004 are “finger-like” in structure, being relatively long and narrow. The use of such flexible circuit board strips may facilitate flexing of the structure 1000, and thus when used in an optical sensor of the type in FIGS. 2A-2B (e.g., by encapsulating the structure 1000 in support structure 206) may facilitate flexing of the optical sensor 200.

Although the configuration of FIG. 10 shows there being two optical sources per flexible circuit board strip 1002 and three optical detectors per flexible circuit board strip 1004, other configurations are possible. One or more optical sources and/or optical detectors may be disposed on each of the flexible circuit board strips.

The circuitry modules 208a-208c may also be disposed on and interconnected by flexible circuitry board strips as shown, and may be coupled to the optical sources and/or optical detectors in this manner.

In some embodiments, such as that shown, the optical sources 202, optical detectors 204, and circuitry modules 208a-208c may each be disposed on a respective rigid circuit board 1006. The respective rigid circuit boards may provide support to the respective components (e.g., to the respective optical sources 202), but in some embodiments may be made no larger than necessary to provide such support and electrical connection to the components, to not negatively impact the flexibility of the structure 1000.

Electrical connection to the respective components (e.g., to the optical sources and optical detectors) may be provided via electrical traces on the flexible circuit board structure, which may make contact with electrical contacts on the respective rigid circuit boards.

The flexible circuit board strips 1002 and 1004 may have any suitable dimensions. Keeping in mind the dimensions previously described as applying to embodiments of the optical sensor 200, the flexible circuit board strips 1002 and 1004 may have lengths (in the x-direction in FIG. 10) of between approximately 30 and 150 mm, between approximately 20 and 50 mm, between approximately 50 and 125 mm, between approximately 60 and 80 mm, approximately 40 mm, approximately 50 mm, approximately 60 mm, approximately 70 mm, approximately 75 mm, or any other suitable value. The flexible circuit board strips may have widths (in the y-direction in FIG. 10) less than approximately 30 mm, less than approximately 20 mm, less than approximately 10 mm, less than approximately 8 mm, less than approximately 7 mm, less than approximately 5 mm, less than approximately 4 mm, or any other suitable value.

In some embodiments, the interspersed pattern of flexible circuit board strips 1002 and 1004 shown in FIG. 10 may be achieved by forming the flexible circuit board strips 1002 and 1004 from a common printed circuit board and then folding one set of flexible circuit board strips relative to the other. FIG. 11 illustrates a non-limiting example of an initial circuit board from which such a structure may be formed.

As shown, the printed circuit board 1100 may include flexible circuit board strips 1002 and 1004 prior to release from the rest of the printed circuit board. A central circuit board segment 1102 interconnects the flexible circuit board strips 1002 and 1004. The central circuit board segment may have any suitable structure, and in the non-limiting example illustrated has a bifurcated structure. Other configurations are also possible.

Folding the printed circuit board 1100 along the line A-A in FIG. 11 may produce the relative positioning of the flexible circuit board strips 1002 and 1004 illustrated in FIG. 10. The optical sources 202, optical detectors 204, and circuit modules 208a-208c may be connected to the flexible circuit board strips 1002 and 1004 prior to or after folding the printed circuit board 1100 along the line A-A. The flexible circuit board strips 1002 and 1004 may be released from the rest of the printed circuit board prior to or after folding of the central circuit board segment along the line A-A.

It should be appreciated that the relative positioning of the flexible circuit board strips 1002 and 1004 illustrated in FIG. 10 can be achieved in manners other than forming the flexible circuit board strips on a common substrate and folding the substrate over. For example, the flexible circuit board strips 1002 may be formed on a first substrate and the flexible circuit board strips 1004 formed on a second substrate. The two substrates may then be aligned and fixed relative to each other in any suitable manner.

FIGS. 12A and 12B illustrate a top view and bottom view, respectively, of a curved optical sensor which may be used in the system of FIG. 1, according to a non-limiting embodiment. The optical sensor 1200 is similar to the optical sensor 200 of FIGS. 2A and 2B, except that instead of having a generally flat configuration in an unbiased state (as for the optical sensor 200), the optical sensor 1200 has a concave (or otherwise curved) configuration in an unbiased state when viewed from the side on which the optical sources and optical detectors are disposed, i.e., the optical sensor 1200 has a curvature to it even when not being forcibly conformed to a subject. In the non-limiting example illustrated, the optical sensor has a curvature about the x-axis, but the curvature could alternatively or additional be around other axes, such as the y-axis. The optical sensor 1200 includes a support structure 1206 which is similar to the support structure 206, previously described in connection with optical sensor 200, except that the support structure 1206 has the curvature illustrated and described. Such curvature may be achieved by suitable molding or other manufacturing techniques. The support structure 1206, like the support structure 206, may be flexible and formed of any other materials previously mentioned in connection with support structure 206, or any other suitable material.

The curved configuration of optical sensor 1200 may be beneficial in conforming to subjects with curved surfaces, such as a head. By providing for curvature in the support structure 1206, less force may be required to conform the optical sensor to the subject to achieve suitable optical coupling. The degree of curvature may be selected in dependence upon the anticipated curvature of subject surfaces to which the optical sensor 1200 is to be coupled. For example, if the optical sensor 1200 is to be placed against a subject's forehead, the degree of curvature may be selected accordingly. Non-limiting examples of suitable radii of curvature include between approximately 10 mm and 200 mm, between approximately 50 mm and 100 mm, any value within such ranges or any other suitable values. As a non-limiting example, the optical sensor 1200 may include curvature around both the x- and y-axes. For example, radius of curvature about the x-axis may be between approximately 100 mm and approximately 150 mm (e.g., 130 mm). The radius of curvature about the y-axis may be between approximately 25 mm and approximately 75 mm (e.g., 50 mm). Other configurations are also possible.

As described previously, an aspect of the present application provides hand-held devices for holding one or more optical sensors of the types described herein. Such hand-held devices may allow for flexibility in placement of an optical sensor and also provide an alternative to a more permanent support. Hand-held devices may be preferable, for example, when short duration optical monitoring is needed (e.g., as a spot check) since they may allow for easy placement of the optical sensor in contact with the subject and then easy removal.

FIGS. 13A-13D illustrate various views of a first non-limiting embodiment of a hand-held device 1300 for holding an optical sensor of the types described herein. FIG. 13A illustrates a front view of the hand-held device 1300, which may also be referred to herein as a hand-held support, a mobile support, or by other similar phraseology. The hand-held device 1300 includes three segments 1302a-1302c, a handle 1304, and anchoring posts or pins 1306 for engaging with the optical sensor.

The three segments 1302a-1302c may be provided to allow for bending or flexing of the optical sensor (not shown). For example, segment 1302a may be hingedly fixed to segment 1302b, and segment 1302b may be hingedly fixed to segment 1302c. In this manner, the three segments may be moved relative to each other, as will be further appreciated by reference to FIGS. 13C-13D. Suitable moving of the segments 1302a-1302c relative to each other may provide a desired degree of curvature (e.g., about the y-axis in FIG. 13A) of the optical sensor, and thus may facilitate conforming the optical sensor to a subject (e.g., the head of a human subject). Moreover, in some embodiments one or more of the segments 1302a-1302c may include pre-curvature about the x-axis in FIG. 13A.

The hand-held device may have any suitable dimensions. According to some embodiments, the segments 1302a-1302c may be sized to accommodate an optical sensor. For example the segments 1302a-1302 may have a combined length (in the x-direction in FIG. 13A) approximately equal to an anticipated length of an optical sensor, and may each have a height (in the y-direction in FIG. 13A) approximately equal to an anticipated height of the optical sensor.

The anchoring posts 1306 represent a non-limiting example of a mechanism for coupling to or otherwise engaging with an optical sensor. For example, the anchoring posts 1306 may alight with alignment holes, corners, notches, or other features of an optical sensor to hold the optical sensor in place. The anchoring posts may have any suitable dimensions for doing so. While four anchoring posts 1306 are shown, it should be appreciated that any suitable number may be provided for suitably engaging with an optical sensor.

Moreover, posts represent a non-limiting example of a mechanism for engaging an optical sensor. Other types of fasteners or couplers may alternatively or additionally be implemented, such as adhesives, straps, elastic bands, hook and loop fasteners, pins, ridges, walls, or other couplers.

The handle 1304 may have any suitable construction. In some embodiments, the handle 1304 may have an ergonomic contour. In some embodiments, the handle may be adjustable in length or angle.

FIG. 13B illustrates a side view of the hand-held device 1300. As can be seen, the segment 1302c may have a curvature to it. Segment 1302a, not visible in FIG. 13B, may likewise have a curvature.

FIG. 13C is a top perspective view of the hand-held device 1300, and shows that the device may include a slider 1308 for adjusting the angle of the segments 1302a and 1302c relative to 1302b, and thus adjusting the curvature of an optical sensor held by the hand-held device 1300. In the non-limiting example shown, the slider may be moved toward and away from the segment 1302b (e.g., by a user's thumb or in any other suitable manner) to adjust the relative positioning of the segments 1302a and 1302c relative to segment 1302b. The slider may locked in a desired position during use to prevent unwanted movement of the segments 1302a-1302c relative to each other.

In the embodiment shown, segments 1302a and 1302c may be moved by the same slider 1308, and thus may be moved in substantially the same manner as each other. However, not all embodiments are limited in this respect. For example, the hand-held device 1300 may be configured to allow for separate (i.e., independent) control of segments 1302a and 1302c.

Furthermore, it should be appreciated that a slider 1308 is a non-limiting example, and that any suitable adjustment mechanism may be used to provide control of the relative positioning of the segments 1302a-1302c. For example, buttons, knobs, or other control or adjustment mechanisms may be used.

FIG. 13D is a top view of the hand-held device 1300.

FIG. 14 illustrates a non-limiting alternative hand-held device for holding an optical sensor of the types described herein. As shown, the hand-held device 1400 includes a base 1402, one or more (e.g., four in this non-limiting embodiment) anchoring posts 1404, one or more (e.g., six in this non-limiting embodiment) compression springs 1406, an anchoring bolt 1408, and a handle 1410.

The base 1402 may have a curvature to provide a desired curvature to an optical sensor held by the hand-held device 1400. The base may be made of plastic or any other suitable material.

The anchoring posts 1404 may engage the optical sensor, and may function in the manner described previously for anchoring posts 1306. The anchoring posts 1404 may be any of the types of fasteners or couplers described previously in connection with anchoring posts 1306.

The compression springs 1406 may apply pressure to the optical sensor to facilitate suitable coupling between the optical sensor and a subject. The springs may be configured to provide any desired degree of pressure. Also, springs represent a non-limiting example of a manner of applying pressure (e.g., local pressure) to the optical sensor and therefore to the subject. For example, air bladders or other compression chambers may additionally or alternatively be implemented.

The anchoring bolt 1408 may facilitate suitable engagement of the hand-held device with the optical sensor and may have any suitable construction for doing so.

The handle 1410 may allow the hand-held device 1400 to be held and manipulated by hand, and may have any suitable construction for doing so.

It should be appreciated that the examples of hand-held supports or devices shown in FIGS. 13A-13D and 14 are non-limiting, and that variations are possible.

According to an aspect of the application, an optical component having a columnar structure is provided. FIGS. 15A-15D illustrate multiple views of two different optical component configurations, either of which may be configured as either an optical source or an optical detector.

As shown in the perspective view of FIG. 15A and the cross-sectional view of FIG. 15B, the optical component 1500 includes a columnar printed circuit board (PCB) 1502 with an upper surface 1504 and a bottom surface 1506, an optically transparent cover 1508, and a sleeve 1510 at least partially surrounding the columnar PCB 1502 and the optically transparent cover 1508. The optical component 1500 may be connected mechanically and/or electrically via a flange 1512 to a support (e.g., a printed circuit board) 1514. A connector 1516 may alternatively or additionally provide electrical coupling between the columnar PCB 1502 and the support 1514.

The columnar PCB 1502 may be formed of any suitable material and may have any suitable shape. In the non-limiting example illustrated, the columnar PCB 1502 has a substantially cylindrical shape with circular cross-section, though other shapes are also possible, such as square cross-sections, multi-sided cross sections, or any other suitable shape. The columnar PCB 1502 may be formed of any suitable material.

In some embodiments, the columnar PCB 1502 may include conductive traces on the upper surface 1504, a non-limiting example of which is shown and described below in connection with columnar PCB 1702 of FIG. 17A. Any such conductive traces (not shown in FIG. 15A) may facilitate making electrical contact to an optically active element disposed on the upper surface 1504. In some such embodiments, conductive paths (e.g., conductive traces, conductive vias, etc.) may extend between the upper surface 1504 and the bottom surface 1506, thus allowing for transmission of electrical signals between the upper surface 1504 and components to which the columnar PCB 1502 may be connected, such as support 1514. Such conductive paths may pass through the columnar PCB (e.g., down the middle of the columnar PCB 1502) or extend along an outer surface of the columnar PCB 1502.

In some embodiments, the columnar PCB may be replaced by a spacer (e.g., lacking any conductive traces) having the same shape and dimensions as the columnar PCB 1502. In such cases, electrical connection from the support 1514 to an optically active element on the spacer may be made using wire leads passing through the spacer, or in any other suitable manner.

Furthermore, it should be appreciated that the columnar PCB 1502 may be any suitable type of PCB, including a fibre-glass sheet PCT (e.g., with copper sheets), a Molded Interconnect Device (MID), or other suitable structure functioning as a PCB. In some embodiments, the columnar PCB 1502 may be formed of a material that is thermally conductive and which may be used for heat dissipation. A ceramic PCB is a non-limiting example.

The optically transparent cover 1508 may serve to cover and protect an underlying optically active element, as will be further illustrated and described in connection with FIGS. 16A, 16C, 17A, and 17D. In some embodiments, the optically transparent cover may perform an optical function, such as focusing outgoing/incoming optical signals (e.g., light). In some embodiments, the optically transparent cover 1508 may function as a light guide, and thus be alternatively referred to as a light guide (e.g., a shaped light guide), or in some embodiments a lens. Its shape may be selected to maximize the light intensity entering the subject from the optical source (when the cover is part of an optical source) or entering the optical detector from the subject (when the cover is part of an optical detector). Thus, the optically transparent cover 1508 may have any suitable shape and may be formed of any suitable material for performing one or more of the described functions.

For example, as shown, the optically transparent cover 1508 may have a substantially cylindrical cross-section (e.g., substantially matching that of the columnar PCB 1502) in some embodiments, and may have a rounded surface (e.g., a dome shape, a half-dome, etc.). However, other geometries are possible, including rectangular cross-sections, among others. Alternative configurations are possible, however, a non-limiting example of which is illustrated in FIG. 19.

As shown in FIG. 19, an optical component 1900 (e.g., an optical source or optical detector) may include the columnar PCB 1502, the sleeve 1510, a light guide 1902 and a pad 1904. The pad may have the substantially flat shape shown, which may be intended to be pressed against a subject. The light guide 1902 and the pad 1904 may be formed of the same material (e.g., both formed of a soft material such as silicone or both formed of a hard material such as polycarbonate) or may be formed of different materials (e.g., the light guide 1902 may be formed of polycarbonate and the pad 1904 formed of silicone). In some embodiments, the light guide may have a reflective coating or material on its walls to facilitate the light guiding functionality. The optical component 1900 may have substantially the same dimensions as those described in connection with optical component 1500, or any other suitable dimensions.

Referring again to FIG. 15A, the optically transparent cover 1508 may be formed of a hard (e.g., non-compressible, such as polycarbonate) or soft (e.g., compressible, such as silicone) material. In some embodiments, a soft material may be selected to improve comfort for the subject, since the optical sources may be forced against the surface of the subject (e.g., being placed in contact with a patient's head). In some embodiments, the optically transparent cover 1508 may be formed of a resin (e.g., a medical grade resin or other biocompatible resin or material). In some embodiments, the optically transparent cover 1508 may include a coating, such as an optically opaque coating applied in a suitable pattern to restrict the angle over which optical signals may be emitted or detected by the optical component. In such embodiments, any suitable coating may be used. In some embodiments, an outer surface of the optically transparent cover may be partially coated with a reflective coating to facilitate the light guiding functionality.

The optically transparent cover may not be transparent to all wavelengths, in some embodiments. In some embodiments, the optically transparent cover may be transparent to optical wavelengths emitted by or detected by an optically active element which the optically transparent cover covers. Thus, in some embodiments the optically transparent cover may have any suitable optical response, including low pass, high pass, and band-pass optical responses. In some embodiments, the optically transparent cover may be transmissive rather than transparent.

The sleeve 1510 may be configured to at least partially surround the columnar PCB 1502 and the optically transparent cover 1508, and in some embodiments the sleeve 1510 is optional. When included, the sleeve 1510 may function as a support for maintaining the relative positioning of the columnar PCB 1502 and the optically transparent cover 1508. The sleeve 1510 may additionally or alternatively perform other functions. For example, the sleeve 1510 may be optically opaque in some embodiments, thus restricting an area or angle over which optical signals can enter/exit the optical component. In some embodiments, the inner wall of the sleeve 1510 may be reflective, for example being coated with a reflective coating. In some embodiments, the sleeve 1510 may be electrically conducting (e.g., formed at least partially of an electrically conductive material, such as having an electrically conductive coating), and may serve as an electrical contact, for example functioning as an electrical ground. Such a configuration is described further below, for example in connection with FIG. 17C. In some embodiments, the sleeve 1510 may be formed of a metal. In some embodiments, the sleeve 1510 may be formed of steel (e.g., AISI 416L steel tube), though other materials may be used.

The flange 1512 may have any suitable configuration for facilitating attachment of the optical component to the support 1514. An adhesive may be used to secure the columnar PCB 1502 to the flange 1512 and to secure the flange 1512 to the support 1514. However, other techniques for attaching the columnar PCB 1502, the flange 1512, and the support 1514 may be used, such as pins, screws, solder bonding, or any other suitable techniques.

The support 1514 may be a printed circuit board providing electrical connection to the optical component 1500, according to a non-limiting embodiment. Thus, support 1514 may include one or more electrical traces thereon in any suitable configuration for connecting to the optical component 1500. In a non-limiting example, the support 1514 may be a rigid printed circuit board.

The connector 1516 may have any suitable configuration for providing electrical interconnection between the optical component 1500 and the support 1514. The connector 1516 may include one or more pins 1518 (e.g., 2 pins, 4 pins, 6 pins, etc.). The pins 1518 may align with electrical contact pads, electrical traces, or other suitable conductive features on the support 1514 or optical component 1500. In some embodiments, the columnar PCB 1502 may include conductive traces on the bottom surface 1506 (not shown in FIGS. 15A-15B), and the pins 1518 (or other suitable connection features of the connector 1516) may make contact with such conductive traces.

The optical component 1500 may have any suitable dimensions. According to an aspect of the present application, an optical component such as that of the type illustrated in FIGS. 15A-15C may be dimensioned to facilitate extending through obstacles, such as hair. Thus, for example, the optical component 1500 may have a relatively small cross-section. Also, the optical component 1500 may be dimensioned so that an optically active element disposed on the upper surface 1504 of the columnar PCB 1502 may be raised above surrounding structures. Such a configuration may be beneficial for a variety of reasons. For example, such a configuration may facilitate close positioning between the optically active element and a subject (e.g., between an LED and a patient's skin), and may minimize interference of the optically active element from surrounding structures.

Some non-limiting examples of suitable dimensions for the optical component 1500 are now provided for purposes of illustration. It should be appreciated that other dimensions are possible, and that the dimensions may be selected based on an intended application of the optical component, for example based on expected obstacles the optical component 1500 may be configured to extend through.

The columnar PCB 1502 may have a height H1 (see FIG. 15B) between the upper surface 1504 and bottom surface 1506 of between approximately 2 mm and 20 mm, between approximately 2 mm and 10 mm, between approximately 3 mm and 7 mm (e.g., 4 mm, 5 mm, or 6 mm), or any other suitable height. The height H2 of the bottom portion of the columnar PCB 1502 may be less than approximately 3 mm, less than approximately 2 mm, less than approximately 1 mm, or any other suitable height. The columnar PCB 1502 may have a width D1 of between approximately 3 mm and approximately 10 mm, between approximately 4 mm and approximately 7 mm, between approximately 0.2 mm and approximately 2 mm, any value within such ranges, approximately 4.5 mm, approximately 5 mm, or any other suitable diameter.

Because of the height H1, the upper surface 1504 and any optically active element disposed thereon may be higher than surrounding structures. For example, the upper surface 1504 is higher than the support 1514. Thus, if the optical component 1500 is positioned adjacent a subject (e.g., contacting the skin of a patient), any optically active element on the upper surface 1504 may be closer to the subject than if the optically active element was directly on, or otherwise closer to, the support 1514. In this manner, optical coupling of the optical component to a subject may be enhanced. Also, interference from surrounding structures may be minimized by elevating an optically active element on the supper surface 1504 above surrounding structures because of the height H1.

The optically transparent cover 1508 may have a width D1 substantially the same as that of the columnar PCB 1502, and thus having any of the dimensions described above in connection with columnar PCB 1502. The optically transparent cover 1508 may have two heights associated therewith, including a height H3 representing the height from the upper surface 1504 to the top of the sleeve 1510 and a total height H4. H3 may be between approximately 0.5 mm and approximately 3 mm, between approximately 1 mm and approximately 2 mm, approximately 1.5 mm, approximately 1.3 mm, or any other suitable value. H4 may be between approximately 1 mm and approximately 6 mm, between approximately 2 mm and approximately 4 mm, approximately 1 mm, approximately 1.5 mm, approximately 2.5 mm, less than approximately 3 mm, or any other suitable value.

The optical component 1500 may have a total height (e.g., H1+H4) of less than approximately 30 mm, less than approximately 10 mm, less than approximately 5 mm, less than approximately 3 mm, between approximately 5 mm and 15 mm, between approximately 2 mm and 6 mm, or any other suitable height.

In some embodiments, the optical component 1500 may be configured such that any optically active element disposed on the upper surface 1504 of the columnar PCB 1502 is located within 5 mm of the top of the optically transparent cover 1508, such that if the optically transparent cover 1508 is placed in contact with a surface of a subject, the optically active element is less than approximately 5 mm from the surface of the subject. In some embodiments, the optical component may be configured such that any optically active element is within approximately 3 mm of the surface of the subject, within approximately 2 mm, or within any other suitable distance.

The sleeve 1510 may have an inner diameter corresponding to the width D1 of the columnar PCB 1502. The sleeve 1510 may have an outer width D2 of between approximately 3 mm and approximately 7 mm, approximately 4 mm, approximately 5 mm, approximately 6 mm, or any other suitable value. In some embodiments, the sleeve 1510, which may represent an outer surface of the optical component 200 as shown in FIG. 15A, may have a cross-sectional area (taken along the line A-A in FIG. 15B) of between approximately 60 mm² and approximately 200 mm², between approximately 80 mm² and approximately 150 mm², approximately 100 mm², approximately 120 mm², approximately 140 mm², or any other suitable cross-sectional area.

It should be appreciated that the dimensions D1 and D2 are referred to herein generally as “widths,” but that they may take more specific forms depending on the shape of the corresponding optical structure. For example, D1 and/or D2 may represent diameters in embodiments in which the columnar PCB 1502, optically transparent cover 1508 and/or sleeve 1510 are cylindrical in nature. However, the columnar PCB 1502, optically transparent cover 1508 and/or sleeve 1510 are not limited to being cylindrical with a circular cross-section. Rather, they may have a square cross-section, a multi-sided cross-section, or any other suitable shapes. In some embodiments, the dimensions D1 and/or D2 may be properly referred to as lengths. Thus, the terminology “width” in this context represents a general identification of a dimension.

The optical component of FIGS. 15A-15B is shown as having substantially constant widths D1 and D2. However, not all embodiments are limited in this respect. For example, FIG. 18 illustrates an alternative configuration, showing a cross-sectional view of an optical component 1800 having a tapered shape. The optical component 1800 has a sleeve 1802 with a width D3 (representing an inner or outer width) that varies along the height H5, such that the optical component is narrower at the end which is to face the subject. Any suitable degree of taper may be implemented, and D3 may fall within any of the ranges previously given for D1 and D2 or within any other suitable ranges. H5 may have any of the values previously described in connection with H1+H4, or any other suitable values. Other configurations are also possible.

FIGS. 15C-15D illustrate an alternative manner of connecting an optical component (e.g., the optical component 1500 of FIG. 15A) to the support 1514. In particular, the configuration of FIG. 15C differs from that of FIG. 15A in that the flange 1512 is omitted. The optical component may be connected to the support 1514 as shown in FIGS. 15C and 15D via solder bonding (e.g., accomplished via suitable solder reflow), epoxy bonding (e.g., gluing with a conductive epoxy), or other similar techniques.

FIGS. 16A-16C illustrate various view of an optical source conforming to the general structure of the optical component 1500 of FIG. 15A. FIG. 16A illustrates an exploded view of the optical source 1600, FIG. 16B illustrates a perspective view of the assembled version of the optical source 1600, and FIG. 16C illustrates a cross-sectional view of the optical source 1600 in assembled form.

As shown, the optical source 1600 includes the columnar PCB 1502, the optically transparent cover 1508, and the sleeve 1510. Multiple optically active elements 1602 are disposed on the upper surface 1504 of the columnar PCB 1502. While four optically active elements 1602 are shown, any number (including one or more, e.g., two, three, eight, or some other number) may be included. In an embodiment, the optical source includes only four optically active elements 1602. The optically active elements 1602 may be emitters (also referred to herein by the terminology “optically emitting elements” and other similar terminology), such as light emitting diodes (LEDs), or any other suitable elements capable of producing optical signals to be emitted from the optical source 1600.

The optically active elements 1602 may electrically couple to the columnar PCB 1502 in any suitable manner. As previously described, the columnar PCB may have electrical contacts, electrical traces, or other suitable electrically conductive features on the upper surface 1504. The optically active elements 1602 may be electrically coupled to such conductive features, for example by solder bonding or in any other suitable manner. Thus, electrical signals (e.g., control signals) may be provided to the optically active elements 1602 via the columnar PCB 1502, for example to control activation of the optically active elements 1602.

As shown in FIG. 16C, each optically active element 1602 may have a height H6, which may take any suitable value. As a non-limiting example, H6 may be between approximately 0.1 mm and approximately 2 mm, between approximately 0.3 mm and approximately 1 mm, approximately 0.3 mm, approximately 0.5 mm, or any other suitable value.

The optical source 1600 may optionally include a filter (not shown) disposed over one or more of the optically active elements 1602. The filter may be any suitable type of filter for passing desired wavelengths from the optically active elements 1602 and blocking other wavelengths. When included, the filter may have any suitable height, for example having any of the heights previously described in connection with H6. In some embodiments, the filter may be implemented as a coating on the optically active elements.

The optical source 1600 may be formed in any suitable manner. According to a non-limiting embodiment, the optically active elements 1602 may be fabricated separately from, and then disposed on, the columnar PCB 1502. The sleeve 1510 may then be positioned around the columnar PCB 1502. A liquid may then be filled into the sleeve 1510 and hardened to form the optically transparent cover 1508. In this way, the sleeve 1510 may function, at least partially, as a mold for formation of the optically transparent cover 208. In some such embodiments, the optically transparent cover 1508 may be formed of a resin (e.g., medical grade resin or other biocompatible resin or material). Alternatively, the optically transparent cover 1508 may be in a solid, preformed state, when disposed in the sleeve 1510. Alternatively, the optically transparent cover 1508 may be disposed on the upper surface 1504 of the columnar PCB 1502 prior to placement of the sleeve 1510 about the columnar PCB 1502. Other manners of making the optical source 1600 are also possible.

The dimensions of the optical source 1600 may take any suitable values, including any of those previously described for the corresponding components in connection with FIGS. 15A-15B. Thus, a detailed discussion of the dimensions is not repeated here.

FIGS. 17A-17D illustrate various views of an optical detector conforming to the general structure of the optical component 200 of FIG. 15A. FIG. 17A illustrates an exploded view of an optical detector 1700. FIG. 17B illustrates a perspective view of the assembled version of the optical detector 1700. FIG. 17C illustrates a connection footprint of the optical detector 1700. FIG. 17D illustrates a cross-sectional view of the optical detector 1700 in assembled form.

As shown, the optical detector 1700 comprises a columnar PCB 1702, the sleeve 1510, a detecting element 1704, and the optically transparent cover 1508. A filter 1706 may also optionally be included, as shown.

The columnar PCB 1702 may be similar to previously described columnar PCB 1502, but is identified by a distinct reference numeral in FIG. 17A because an example of conductive traces is also illustrated. Namely, the columnar PCB 1702 may include a first conductive trace 1708 toward to the base of the columnar PCB 1702 and a conductive trace pattern 1710 on an upper surface of the columnar PCB 1702. The conductive trace 1708 and the conductive trace pattern 1710 may allow for transmission of electrical signals between the optical component and other structures, such as support 1514.

FIG. 17C illustrates a non-limiting example of a connection footprint for connecting an optical component (e.g., optical component 1500) to a support (e.g., support 1514), such as a rigid PCB or other support. The illustrated footprint may be used, for example, when the optical component is to be soldered to the support 1514. It should be appreciated that the illustrated trace pattern is a non-limiting example.

In a non-limiting embodiment, the sleeve 1510 may contact the conductive trace 1708 when the optical detector is assembled. The conductive trace 1708 may function as an electrical ground contact, and thus in a non-limiting embodiment the sleeve 1510 may be electrically grounded. The conductive trace pattern 1710 may be suitable for coupling to and communicating electrically with the detecting element 1704. For example, the detecting element may include a corresponding electrical trace pattern or pin configuration, as non-limiting examples, configured to align with the conductive trace pattern 1710. Other patterns than that represented by conductive trace pattern 1710 may alternatively be used, as the conductive trace pattern 1710 is a non-limiting example provided for purposes of illustration.

The columnar PCB 1702 may include conductive paths (e.g., conductive traces, conductive vias, etc.) between the conductive trace pattern 1710 and the bottom surface of the columnar PCB, thus allowing for transmission of electrical signals between the conductive trace pattern 1710 and components to which the columnar PCB 1702 may be connected, such as support 1514. Such conductive paths may pass through the columnar PCB 1702 (e.g., down the middle of the columnar PCB 1702) or extend along an outer surface of the columnar PCB 1702.

The detecting element 1704 may be any suitable type of detecting element for detecting desired optical signals (e.g., optical signals in a wavelength range of interest). In some embodiments, the detecting element 1704 may produce an electrical signal indicative of the intensity, phase, and/or frequency of detected optical signals. The detecting element 1704 may be a photodetector (e.g., a pin photodetector, a phototransistor, a silicon photodetector, or an infrared photodetector, as non-limiting examples). As shown in FIG. 17B, the detecting element 1704 may be centered on an upper surface of the columnar PCB 1702 when the optical detector 1700 is assembled.

As described, the optical detector 1700 may optionally comprise a filter 1706. The filter 1706 may filter out undesired wavelengths from any optical signals received by the optical detector 1700. According to a non-limiting embodiment, the filter 1706 may be a color filter, though other types of filters are also possible. The filter 1706 may be suitably positioned with respect to the detecting element 1704 to perform the filtering function. For example, the filter 1706 may be disposed on, and centered with respect to, the detecting element 1704 according to a non-limiting embodiment. In some embodiments, the filter 1706 may be implemented as a coating on the detecting element.

The optically transparent cover 1508, previously described, may be disposed on the columnar PCB 1702 and may cover the detecting element 1704 and filter 1706, as shown in FIG. 17D.

The optical detector 1700 may be formed in any suitable manner. According to a non-limiting embodiment, the detecting element 1704 may be fabricated separately from, and then disposed on, the columnar PCB 1702. Optionally, the filter 1706 may be disposed on the detecting element 1704. The sleeve 1510 may then be positioned around the columnar PCB 1702. A liquid may then be filled into the sleeve 1510 and hardened to form the optically transparent cover 1508. In this way, the sleeve 1510 may function, at least partially, as a mold for formation of the optically transparent cover 1508. In some such embodiments, the optically transparent cover 1508 may be formed of a resin (e.g., a medical grade resin or other biocompatible resin or material). Alternatively, the optically transparent cover 1508 may be in a solid, preformed state, when disposed in the sleeve 1510. Alternatively, the optically transparent cover 1508 may be disposed on the columnar PCB 1702 prior to placement of the sleeve 1510 about the columnar PCB 1702. Other manners of making the optical detector 1700 are also possible.

The optical detector 1700 may have any suitable dimensions. Referring to FIG. 17D, several of the illustrated dimensions have been previously described herein, and the values for such dimensions apply in the context of the optical detector 1700 as in the context of an optical source (e.g., optical source 1600), or an optical component more generally (e.g., optical component 1500). The detecting element 1704 may have a height H7 of a value given by any of those values previously described for height H6, a height H7 of less than approximately 1 mm, or any other suitable values. The filter 1706 may have a height H8 given by any of those values previously described in connection with height H6, a height H8 of less than approximately 2 mm, less than approximately 1 mm, or any other suitable values.

Optical components according to aspects of the present application may be operated in any suitable manner, as the manner of operation is not limiting. For example, optical source 1600 and optical detector 1700 may be operated in any suitable manner to emit and detect, respectively, optical signals.

Optical components according to aspects of the present application may operate at any suitable wavelengths. Thus, optical sources (e.g., optical source 1600) may emit (via optically active element 1602) any suitable wavelengths of optical radiation. In some embodiments the optical sources may operate at any of the wavelengths described previously in connection with optical sources 202.

Optical detectors according to aspects of the present application, such as optical detector 1700, may detect the wavelengths emitted by the optical sources. Thus, for example, optical detector 1700 may detect any of the wavelengths previously described as being emitted by an optical source. In some embodiments, a filter of a detector (e.g., filter 1706) may select out certain wavelengths reaching a detecting element of an optical detector.

Optical components of the types described herein may be used in various contexts. For example, the optical components of the types described herein may be used in optical sensors 200 and in the system of FIG. 1.

It should be appreciated from the foregoing that optical components according to various aspects of the present application may be used to emit and/or detect optical signals sent into and received from a subject's head. Detection of such optical signals may provide information relating to the subject, which may be useful, for example, in detecting and/or analyzing a physical condition of a subject (e.g., a patient's brain).

While system 100, and the sensor 104, represent a non-limiting example of systems and apparatus which may utilize optical components of the types described herein (e.g., optical component 1500, optical source 1600, optical detector 1700, etc.), it should be appreciated that optical components according to the various aspects of the present application are not limited to being used in such systems and apparatus. Thus, other uses for optical components according to aspects of the present application are also possible.

Applicants have appreciated that, in the context of performing diffuse optical tomography (DOT) measurements on a subject, it may be desirable to gather and/or analyze information about more than two physical characteristics or conditions of the subject. For example, when considering a human subject, it may be desirable to gather and/or analyze information relating to endogenous biological chromophores (e.g., oxygenated hemoglobin; de-oxygenated hemoglobin; lipids; water; myoglobin; bilirubin; and/or cytochrome C oxidase) and/or exogenous chromophores (e.g., indocyanine green (ICG) or other biologically compatible near infrared (NIR) absorbing optical dyes or tracers). Applicants have further appreciated that, in performing DOT investigations of a subject, the desire to gather information about more than two physical characteristics or conditions may be achieved by using more than two wavelengths, and furthermore that suitable positioning of optical sources and detectors allows for substantially the same spatial portion of a subject to be investigated using the different wavelengths.

Thus, according to an aspect of the application, a diffuse optical tomography (DOT) sensor includes a plurality of optical sources disposed at respective locations of the sensor. Each optical source of a first subset of the optical sources may be configured to produce or emit a first plurality of optical signals with a first plurality of center wavelengths and each optical source of a second subset of the optical sources may be configured to produce a second plurality of optical signals with a second plurality of center wavelengths. The first and second pluralities of center wavelengths may be different than each other, and thus the DOT sensor may produce optical signals of more wavelengths than are produced by any single optical source of the DOT sensor. In a non-limiting embodiment, each optical source of the first subset may produce optical signals of four center wavelengths and likewise each optical source of the second subset may produce optical signals of four center wavelengths different than the four center wavelengths produced by the optical sources of the first subset.

The DOT sensor may also include a plurality of optical detectors disposed at respective locations. The optical detectors may have suitable detection capabilities to be capable of detecting any of the wavelengths emitted by any of the optical sources. The optical detectors may be positioned relative to the first and second subsets of optical sources such that substantial spatial overlap occurs in the paths of the optical signals traversed from the first subset of optical sources to the optical detectors and the second subset of optical sources to the optical detectors. In this manner, substantially the same spatial area may be investigated using the first plurality of center wavelengths and the second plurality of center wavelengths. In a non-limiting embodiment, the optical sources and the optical detectors may collectively form an array.

In some embodiments, the subject may be a human patient and a target area of study may be the patient's brain, although other subjects and/or target areas of interest may be studied (e.g., a limb, a torso, skin flap, organ, breast, tissue exposed by surgery, or other region of interest). In such situations, it may be desirable to monitor multiple physical characteristics of the brain.

As described already, the use of multiple wavelengths when investigating a subject with an optical sensor (such as a DOT sensor) may facilitate investigation of multiple physical characteristics of a subject to which the DOT sensor is optically coupled. For example, the first or second pluralities of center wavelengths may be used to provide information about absorption or scattering within a subject. For example, the first or second pluralities of center wavelengths may be used to provide information about absorption of hemoglobin (oxygenated or deoxygenated) in the subject, absorption of lipids in the subject, absorption of water in the subject, or scattering behavior within the subject. For example, in some embodiments each wavelength of the first and second pluralities of center wavelengths may provide information about both absorption and scattering within the subject. In some embodiments, the first plurality of center wavelengths and/or the second plurality of center wavelengths may provide redundant information, i.e., information about the same physical characteristic (e.g., deoxygenated hemoglobin) of the subject as a different wavelength of the first or second pluralities of center wavelengths. Such redundancy may, for example, increase confidence in collected data related to a particular physical characteristic. Suitable processing of detected optical signals (e.g., provided by an optical detector of an optical sensor) may facilitate derivation of information relating to any of the above-listed items.

According to an aspect of the application, a method of operating an optical sensor such as sensor 200 is provided. The optical sensor may include a plurality of optical sources and a plurality of optical detectors. The optical sources may be controlled to irradiate a subject (e.g., a patient) with optical signals. According to an aspect, different optical sources of the optical sensor may emit different pluralities of center wavelengths, thus allowing for analysis of multiple different physical characteristics or conditions of the subject. The optical signals may pass through the subject and be detected by the optical detectors upon exit from the subject. In some embodiments, the optical signals from the sources may enter the subject and cause an optical emission within the subject that is then detected by the detectors.

For example, in some embodiments the optical sources of the optical sensor 200 need not all emit the same wavelengths. For example, a first optical source may emit a first wavelength (e.g., approximately 650 nm) and a second optical source may emit a second wavelength (e.g., approximately 800 nm). In fact, aspects of the application provide for different optical sources to emit different pluralities of wavelengths. Recognizing that in practice many optical emitters, such as LEDs, emit a spectrum of frequencies, be it narrowband or broadband, aspects of the application provide for different optical sources to emit different plurality of center wavelengths. By utilizing multiple optical sources to emit a greater number of wavelengths than would be possible or practical with a single optical source, information may be gathered relating to a greater number of physical characteristics of a subject than would be possible or practical with a single optical source. Also, the use of multiple wavelengths may facilitate detection of various quantities of interest with respect to the subject since different wavelengths of the radiation may behave differently when passing through the subject. A non-limiting example is now described in the context of FIG. 4, though it should be appreciated that the aspects described herein relating to emitting different pluralities of center wavelengths from different optical sources may apply to other optical sensor configurations as well.

According to a non-limiting embodiment, optical source 1 in FIG. 4 may emit a first plurality of wavelengths, and may have any suitable structure for doing so. For example, optical source 1 may emit wavelengths of 650 nm, 700 nm, 750 nm, and 800 nm. In some embodiments, the listed wavelengths represent center wavelengths, to be distinguished from a scenario in which the optical source is a broadband emitter covering the wavelengths listed. Optical source 2, according to a non-limiting embodiment, may emit a second plurality of wavelengths different than those emitted by optical source 1, and may have any suitable structure for doing so. For example, optical source 2 may emit wavelengths of 850 nm, 900 nm, 925 nm, and 950 nm. Again, the listed wavelengths may represent center wavelengths rather than a single broadband emission encompassing the listed wavelengths.

In some embodiments, the different center wavelengths emitted by different optical sources may be “interleaved” with respect to each other. For example, a first optical source may emit wavelengths of 650 nm, 750 nm, 850 nm, and 925 nm, while a second optical source may emit wavelengths of 700 nm, 800 nm, 900 nm and 950 nm. Other manners of dividing the center wavelengths between two or more optical sources are also possible.

It can be seen from the above-described examples that optical source 1 may emit four different (center) wavelengths than optical source 2. The remaining optical sources of the optical sensor 200 may similarly be split between those that emit the first plurality of wavelengths and those that emit the second plurality of wavelengths. For example, optical sources 1, 4, 5, 8, and 9 may represent a first subset of optical sources in which each emits the first plurality of wavelengths, while optical sources 2, 3, 6, 7, and 10 may represent a second subset of optical sources in which each emits the second plurality of wavelengths. In this manner, the optical sensor 200 may operate with a greater number of wavelengths than produced by any single optical source of the optical sensor.

In those embodiments in which different optical sources of an optical sensor emit different pluralities of wavelengths, such as the non-limiting embodiment just described, the optical sources may be arranged in any suitable configuration in combination with the optical detectors such that the same spatial area of a subject may be investigated with the different wavelengths. Referring still to FIG. 4, optical signals from optical source 1 may be detected by, for example, optical detectors 1-9. Likewise, optical signals from optical source 2 may be detected by, for example, optical detectors 1-9. Thus, even though optical source 1 and optical source 2 are disposed at different positions (or locations) of the optical sensor, there may be substantial overlap in the paths between those two optical sources and the optical detectors which detect the optical signals from those two optical sources. As a result, the same spatial area of the subject may effectively be investigated by the first and second pluralities of wavelengths even though optical source 1 and optical source 2 are at different locations. Suitable arrangement of the remaining optical sources and optical detectors of the optical sensor may likewise provide for effectively the same spatial coverage from the different wavelengths emitted by the different optical sources.

While the above-described example identifies optical sources 1, 4, 5, 8, and 9 as emitting the same wavelengths as each other and optical sources 2, 3, 6, 7 and 10 as emitting the same wavelengths as each other, it should be appreciated that other configurations are possible. For example, optical sources 1, 3, 5, 7, and 9 may represent a first subset of optical sources in which each emits the first plurality of wavelengths and optical sources 2, 4, 6, 8, an 10 may represent a second subset of optical source in which each emits the second plurality of wavelengths. Other configurations are also possible.

Moreover, it should be appreciated that more than two subsets of optical sources may be provided in which the subsets emit different wavelengths than the other subsets. For example, three, four, and any number of subsets of optical sources emitting respective pluralities of wavelengths may be provided.

In the non-limiting example described above, optical source 1 and optical source 2 each emit four (center) wavelengths. It should be appreciated that any suitable number of two or more wavelengths (e.g., two, three, four, five, six, eight, ten, etc.) may be emitted, and that four represents a non-limiting example. For instance, optical source 1 may emit a first plurality of wavelengths comprising two or more first wavelengths and optical source 2 may emit a second plurality of wavelengths comprising two or more second wavelengths. Also, the optical sources need not emit the same number of wavelengths. For instance, optical source 1 may emit two center wavelengths and optical source 2 may emit three center wavelengths. Other numbers are also possible.

In some embodiments, a first optical source of an optical sensor emits a first plurality of center wavelengths consisting of two first center wavelengths and a second optical source of an optical sensor emits a second plurality of center wavelengths consisting of two second center wavelengths different than the two first center wavelengths. In some embodiments, a first optical source of an optical sensor emits a first plurality of center wavelengths consisting of four first center wavelengths and a second optical source of an optical sensor emits a second plurality of center wavelengths consisting of four second center wavelengths different than the four first center wavelengths. Other configurations are also possible.

For example, in some embodiments two optical sources may emit different pluralities of center wavelengths but may exhibit some overlap in the center wavelengths emitted. For example, two optical sources may each emit 750 nm and 800 nm, but one of the two optical sources may also emit 650 nm and 700 nm while the other optical source may also emit 850 nm and 900 nm. Other manners of partial overlap of the center wavelengths emitted by different optical sources are also possible.

It should also be appreciated that the center wavelengths listed above in the context of FIG. 4 (i.e., 650 nm, 700 nm, 750 nm, and 800 nm for optical source 1 and 850 nm, 900 nm, 925 nm, and 950 nm for optical source 2) are non-limiting examples, and that any suitable center wavelengths may be used. For example, any center wavelengths within the wavelength ranges previously described (e.g., between 600 nm and 1000 nm) may be used.

The optical detectors may detect the wavelengths emitted by the optical sources. In some embodiments, all the optical detectors may be capable of detecting any of the wavelengths emitted by any of the optical sources. In such embodiments, all the optical detectors may be substantially identical to each other. However, in some embodiments different optical detectors may be capable of detecting different wavelength ranges from each other (e.g., due to different types of optical detecting elements or different filtering schemes, among other possibilities).

In addition to using different wavelengths, aspects of the present application provide for use of different optical intensities. For example, two optical sources emitting the same center wavelengths as each other may do so with different intensities. The different intensities may be used, for example, to improve signal-to-noise ratio (SNR) for the optical sources. In fact, in some scenarios it may be necessary to use different intensities for different wavelengths to improve SNR.

In some embodiments, the optical sensor 200 may be used to provide information about the concentration and oxygenation of hemoglobin in a subject (e.g., the concentration and oxygenation of hemoglobin in a subject's brain, muscle or other tissues). Thus, the wavelengths of radiation used by the optical sensor 200 may be selected to facilitate collection of such information. In some embodiments, the wavelengths utilized by the optical sensor 200 may be approximately equally dispersed over the range from approximately 650 nm to approximately 950 nm. A broader spectrum may be used at the higher end of this range, in some embodiments. A narrower range (i.e., narrower than 650 nm to 950 nm) may be used in some embodiments, for example those embodiments in which only two to four wavelengths are to be used. In some embodiments, only two wavelengths may be used, with one below the isosbestic point of hemoglobin, which is about 800 nm, and one above (e.g., one wavelength below approximately 765 nm and one wavelength above approximately 830 nm).

As previously described, a plurality of different wavelengths may be used by the optical sensor 200 to gather information relating to different characteristics of a subject (such as a human patient). In general, the use of N wavelengths may provide information about N targets (e.g., N chromophores). For example, the N targets may have respective absorption or scattering coefficients, and thus use of N different wavelengths may allow for solving for the N coefficients. In some embodiments, more than N wavelengths may be implemented by an optical system to determine the N coefficients, such that the solution for the N coefficients may be over-determined. Such a technique may be used to provide redundancy of information and/or a more robust solution.

As a non-limiting example, the use of two different wavelengths may provide information about absorption of oxygenated hemoglobin in the subject and absorption of deoxygenated hemoglobin in the subject. Use of an additional third wavelength may provide information about absorption of lipids in the subject, in addition to the information about absorption of oxygenated and deoxygenated hemoglobin. Use of an additional fourth wavelength may provide information about absorption of water in the subject in addition to the information about oxygenated and deoxygenated hemoglobin and lipids. Use of additional fifth and sixth wavelengths may provide information about scattering within the subject in addition to the types of information previously described. Thus, according to embodiments of the present application, first and second pluralities of wavelengths may in combination include N or more wavelengths to provide, in combination, information about absorption and/or scattering of N targets (e.g., any of those targets previously described). In some embodiments, four total wavelengths may be used, five total wavelengths, six total wavelengths, seven total wavelengths, eight total wavelengths, or any other suitable number.

Such wavelengths may be suitably selected based on the target (e.g., lipids, hemoglobin, etc.), and may be divided among optical sources of the optical sensor in any suitable manner. For example, a first optical source (e.g., optical source 1 in FIG. 4) may emit wavelengths to provide information about absorption of oxygenated and deoxygenated hemoglobin, absorption of lipids, and absorption of water. A second optical source (e.g., optical source 2 in FIG. 4) may emit wavelengths to provide information about scattering of particular targets within a subject.

In some embodiments, one or more wavelengths may be used by an optical sensor (e.g., optical sensor 200) to provide redundant information. For example, one or more wavelengths may provide redundant information to one or more other wavelengths used by the optical sensor. Such redundancy may be desirable to provide increased confidence in collected data with respect to a given target, to provide a backup data channel in the event a particular wavelength proves ineffective, or for any other reason.

Suitable processing of detected optical signals (e.g., provided by an optical detector of an optical sensor) may facilitate derivation of information relating to any of the above-listed items. Such processing may be performed, for example, by a host module 106 and/or central unit 108 of a system such as that of FIG. 1. An optical detector of an optical sensor may detect the various wavelengths emitted by the plurality of optical sources and may provide resulting signals to the host module 106 and central unit 108 for processing. Other manners of processing detected optical signals are also possible.

An optical sensor having optical sources configured to emit different wavelengths or different pluralities of wavelengths may be operated in various manners including any of those described herein. For example, the operation described previously in connection with FIG. 9 may be implemented, optical detectors 204 may sample simultaneously.

As previously described, in some embodiments two or more (and in some cases, each) optical source of an optical sensor may emit a plurality of (center) wavelengths. Thus, considering the operation described in FIG. 9, and assuming that each of the optical sources 1-10 in that non-limiting example emits a plurality of wavelengths, optical source 1 may emit a plurality of wavelengths (e.g., four center wavelengths) during time slot 902. The plurality of wavelengths of optical source 1 may be emitted sequentially, concurrently, substantially concurrently, or substantially simultaneously within time slot 902.

As used herein, the emission of two signals is concurrent if the signals have any overlap in time as they are being emitted. Depending on the context, the emission of signals is substantially concurrent if overlapping in time by at least 80%, by at least 90%, or more. In some embodiments, signals may be emitted generally serially such that a first one or more signals is concurrent with a second one or more signals, the second one or more signals is concurrent with a third one or more signals, etc., even though the third one or more signals may or may not be concurrent with the first one or more signals. The emission of two signals is substantially simultaneous if overlapping in time by approximately 95% or more.

The operation previously described with respect to time slots 904, 906, and 908 may then be performed. Subsequently, the second optical source may be activated and the plurality of wavelengths from that optical source may be emitted sequentially, concurrently, substantially concurrently, or substantially simultaneously. Demodulation, packetization and transfer, and buffer time slots may then be observed, before proceeding to the third optical source. The process may continue until all the optical sources have been activated.

It should be appreciated that in an alternative embodiment demodulation of sampled signals from a first optical source, and packetization and transfer of data for the first optical source may occur in parallel to sampling of signals from a second optical source. Thus, the aspects described herein are not limited to a particular manner of timing sequence.

It should be appreciated from the foregoing that an aspect of the application provides a method of operating a diffuse optical tomography (DOT) sensor, comprising emitting, into a subject from a first optical source located at a first position of the DOT sensor, a first plurality of (center) wavelengths substantially concurrently during a first time interval and detecting the first plurality of wavelengths from the first optical source during the first time interval with first and second optical detectors located at second and third positions, respectively, of the DOT sensor. In some embodiments, the distance between the first position and the second position is less than a distance between the first position and the third position. For example, the first optical source and the first optical detector may be first nearest neighbors, and the first optical source and second optical detector may be second nearest neighbors.

The method may further include emitting, into the subject from a second optical source located at a fourth position of the DOT sensor, a second plurality of (center) wavelengths different than the first plurality of (center) wavelengths substantially concurrently during a second time interval. The first and second time intervals may be non-overlapping. The second plurality of (center) wavelengths from the second optical source may be detected with the first and second optical detectors of the DOT sensor.

The method may further include emitting, into the subject from a third optical source located at a fifth position of the DOT sensor, the first plurality of (center) wavelengths substantially concurrently during a third time interval. The third time interval may be non-overlapping with the first time interval and/or the second time interval. The first plurality of (center) wavelengths emitted from the third optical source may be detected during the third time interval with the first and second optical detectors.

In some embodiments, such as the non-limiting embodiment of FIG. 4, the first, second, and third optical sources and the first and second optical detectors collectively form at least part of an array of optical sources and optical detectors. As described previously, suitable positioning of the optical sources and optical detectors with respect to each other may allow for substantially the same spatial area to be investigated with the different pluralities of wavelengths, despite the different pluralities of wavelengths being emitted by optical sources located at different positions.

In some embodiments in which a method like that described above is implemented, only one optical source may be activated at any given time, and thus the wavelengths emitted by that optical source may be the only wavelengths emitted during that particular time interval. However, not all embodiments are limited in this respect. In some embodiments, for example, multiple optical sources may be activated at the same time.

Aspects of the present application relate to supports for supporting an optical sensor in a desired position with respect to a subject, for example, for use as support 102 in FIG. 1. In some embodiments, the supports may be suitable to support an optical sensor in close proximity to, or in contact with, a subject's head, and in some such embodiments may represent a headpiece or brain cap. The supports may have a multi-piece configuration, with the multiple pieces being attachable to each other to form a closed contour (or substantially closed contour), such as a closed loop around the subject's head. The sizing of the loop may be adjustable and mechanisms may be provided as part of the support for adjusting the pressure with which the optical sensor(s) supported by the supports contacts the subject's head.

In some embodiments, the supports may feature an open-top construction, allowing access to a desired part of a subject, such as the top of the subject's head, the area around a subject's ears, and/or the temporal region above a subject's cheekbone (the zygmotic arch). Thus, the multiple pieces of the support may be interconnected to form a loop around a chosen portion of the subject (e.g., the subject's head) without obstructing the portion desired to remain accessible (e.g., the top of the subject's head, the area around a subject's ears, and/or the temporal region above a subject's cheekbone). The supports may be removed by detaching (or disengaging/decoupling) the multiple pieces, without obstructing the portion of the subject desired to remain accessible. In this manner, the supports may be applied and removed without obstructing the portion of the subject desired to remain accessible, and therefore without obstructing any objects (e.g., medical instruments) in place on the portion of the subject desired to remain accessible.

In some embodiments, the supports may be disposable. The optical sensors may be used to analyze various subjects including medical patients. The supports may contact the subject (e.g., the medical patient), and therefore become soiled, contaminated, aesthetically unappealing, or otherwise impacted in a manner such that it may be desirable to dispose of and replace the support when using the optical sensor on a different subject, or even at various points in time during use of an optical sensor on the same subject. Thus, in some embodiments the supports may be disposable in nature, for example being formed of relatively inexpensive materials and being easily attached to or detached from one or more optical sensors. Thus, while a single optical sensor may be used in conjunction with multiple subjects, a support according to aspects of the present application may be disposed of after use on a single subject or multiple supports may be used on a single subject in turn and discarded.

The support may include multiple pieces of flexible and/or soft material which may be suitably attached to apply the optical sensor to the subject and which may be detached or disengaged from each other to remove the optical sensor from the subject. In some embodiments, the support may be configured to support an optical sensor against (or in contact with) a subject's head (e.g., in contact with a human patient's head). The support may include at least two distinct segments, which in some embodiments may be cushions and/or straps. A first segment (or cushion in some embodiments) may be configured to engage with (or couple to or contact) a back portion and, optionally, side portions of the subject's head. For example, the first segment may engage with a subject's occiput. A second segment (or cushion) may be configured to engage with (or couple to or contact) front and, optionally, side portions of the subject's head. For example, the first segment may be an elongated strip which wraps from one side of the subject's head around the front of the subject's head to the opposing side of the subject's head. FIGS. 20A-20C illustrate a non-limiting example.

FIG. 20A is a front view of a support 2000 engaged with a subject. In particular, in the non-limiting example shown, the subject is a human head 2002, having a back or rear portion 2004, a front portion (e.g., the forehead) 2006, and sides 2008.

As shown in FIGS. 20B and 20C, which are a top view and a front perspective view, respectively, the support 2000 includes two segments, or pieces, 2010 and 2012. The first segment 2010 is engaged with the back or rear portion 2004 of the head 2002. The second segment 2012 is engaged with the front portion 2006 and sides 2008 of the head 2002.

As shown, the support 2000 has an open-top construction, such that the support 2000 engages with the head 2002 in a manner which leaves the top of the head 2002 unobstructed (or uncovered) by the support. Such a configuration may be desirable in circumstances in which access to the top of the head 2002 is desirable or necessary, for example when a doctor needs access to the top of the head 2002 to perform a procedure or evaluate the head 2002. Moreover, the support may allow unimpeded physical access to the area around the subject's ears, and/or the temporal region above the subject's cheekbone.

The first and/or second segments 2010 and 2012 may be formed of any suitable materials. In some embodiments, it may be desirable for the first and/or second segments 2010 and 2012 to be configured to flex or otherwise conform to the subject. For example, as shown, the first segment 2010 may conform to the back 2004 of the head 2002 and the second segment 2012 may be configured to conform to the front 2006 and sides 2008 of the head 2002. In some embodiments, the first and/or second segments 2010 and 2012 may be configured to flex in at least two orthogonal directions, such as the x and y-directions shown in FIG. 20B. By making the first and/or second segments 2010 and 2012 conformable to the subject's head, proper placement of an optical sensor supported by the support against the subject's head may be achieved. Thus, the first and/or second segments may be formed of materials that are conformable, deformable, flexible, or malleable, in some embodiments.

In some embodiments, the first and/or second segments 2010 and 2012 may be formed at least in part of materials suitable for use on a human subject and, in some instances, for use in a medical setting. For example, the first and/or second segments 2010 and 2012 may be formed of a soft material or cushioning material (e.g., foam (e.g., memory foam, laminated foam, polyurethane foam or other suitable foam), cloth, fabric, polyester, rubber, a combination of such materials, or any other suitable material) which may render the support 2000 more comfortable to the subject or wearer, as well as facilitating the ability of the support to conform to the subject, as described above. In some embodiments, the first and/or second segments 2010 and 2012 may be formed at least in part of a breathable material, wicking material, or other suitable material, for example to improve air flow and reduce moisture (e.g., sweat) retention. In some embodiments, the first segment and/or second segment 2010 and 2012 may be formed at least in part of a material exhibiting antimicrobial properties, stain removal properties, mildew resistance, or other properties, which may be important for example when the support is used on a subject with open wounds or other potentially harmful medical conditions. In some embodiments, the first segment and/or second segment may comprise medical grade fabric.

As shown in FIGS. 20A and 20B, the first and second segments 2010 and 2012 may be interconnected in any suitable manner to hold them in place on the head 2002 (or subject more generally). In some embodiments, one or more first mechanisms may be provided to connect the first segment 2010 and second segment 2012 in a closed contour which may be fitted to the head 2002 (or subject more generally). For example, a hook and loop fastener may be included (e.g., with suitable components on the first segment 2010 and second segment 2012) to allow for the first segment 2010 and second segment 2012 to be connected in a loop. In some embodiments, or one more second mechanisms may be provided to size, tighten, or tension the support 2000 about the head 2002. For example, a strap (e.g., an elastic strap), band, string, or other mechanism may be provided. Non-limiting examples of such feature are described further below.

The first segment 2010 and second segment 2012 may take any of various suitable configurations, which to at least some extent may depend on the manner in which the support is to be used. An example of a suitable first segment 2010 for engaging with a subject's head is shown in FIGS. 21A and 21B.

FIG. 21A illustrates an inner surface of a support segment 2100 which may be used as the first segment 2010 in the support 2000 of FIG. 20A, i.e., FIG. 21A illustrates the surface of the segment 2100 configured to face the subject when the segment 2100 is engaged with the subject. FIG. 21B illustrates an outer surface of the support segment 2100, i.e., the surface of the segment 2100 which faces away from the subject when the segment 2100 is engaged with the subject.

As shown, the segment 2100 may include a body (or support or substrate) 2102, which may be a cushion in some embodiments. A strap 2104 is fastened (or affixed or anchored) to an upper part of the body 2102, on the outer surface as shown in FIG. 21B. The strap 2104 may be fastened by stitching 2105 or in any other suitable manner. The segment 2100 further comprises straps 2106a and 2106b, which may be fastened or anchored to lower portions 2108a and 2108b, respectively. The straps 2106a and 2106b may be fastened to the body 2102 on the outer surface, as shown in FIG. 21B, by stitching 2107 or other suitable fastener.

The body 2102 may be soft and/or conformable, for example to facilitate conforming of the segment 2100 to a subject. Thus, the body 2102 may be formed of any suitable material described herein for a support (e.g., foam (e.g., memory foam, laminated foam, polyurethane foam, or other suitable foam), cushioning, rubber, knit spacer material, fabric, polyester, any combination of those materials), or any other suitable material. Moreover, the lower portions 2108a and 2108b may be able to bend (or flex or fold) about the lines 2110a and 2110b, respectively, relative to the body 2102. For example, the lower portions 2108a and 2108b may be formed of distinct foam pads from the rest of body 2102, attached by stitching (e.g., the lines 2110a and 2110b may represent a physical structure forming a flex point such as stitching in some embodiments) or other delineating feature. Alternatively, the body 2102 may include a single structure (e.g., a single foam pad) with a suitable feature placed at the locations of lines 2110a and 2110b to make lower portions 2108a and 2108b distinctly flexible relative to the remainder of the body 2102.

The straps 2104, 2106a and 2106b may function to connect the segment 2100 to another segment of a support (e.g., second segment 2012 in FIG. 20A). An example of such interconnection is described further below in connection with FIGS. 23A, 23B, and 26. In this manner, the multiple segments may be formed into a closed contour for engaging the support with the subject. The straps 2104, 2106a, and 2106b may be any suitable straps, including elastic straps, and may have any suitable dimensions. In some embodiments, the straps may include features facilitating their connection to another component. For example, fasteners (e.g., hook and loop features)2116a, 2116b, 2118a, and 2118b may be included. The fasteners 2116a, 2116b, 2118a, and 2118b may be suitably positioned on an appropriate surface of the straps to facilitate their intended connection to other components. It should be appreciated that while straps 2104, 2106a, and 2106b are shown as part of segment 2100, other connectors and fasteners may alternatively be used.

The segment 2100 may optionally include an indicator feature or alignment feature for providing an indication of the positioning of the segment. For example, an indicator 2114 may be provided as shown in FIG. 21B, and may represent a sticker, a colored portion of the segment, colored stitching, a notch in the segment, a bump, or other indicator. A user applying the segment 2100 to a subject may use the indicator to align the segment, for example by positioning the indicator centrally over the rear portion of the subject's head. Depending on the nature of the indicator 2114, it may or may not be visible on the inner surface of the segment 2100 and therefore is shown in dashed lining in FIG. 21A.

FIG. 22 illustrates an example of a suitable segment of a support which may be used as a second segment 2012 in FIG. 20A. As shown, the segment 2200 may generally be in the shape of an elongated strip, having a length L4 and a width W1, though other shapes are also possible. The segment 2200 may be divided conceptually, and in some embodiments physically, into multiple portions 2202a-2202c. Each portion may hold or engage with a respective optical sensor in some embodiments. Thus, the segment 2200 may be configured to hold three optical sensors, though it should be appreciated not all embodiments are limited in this respect. For example, support segments may be configured to hold one or more optical sensors, and in some embodiments certain support segments may not hold any optical sensors. For instance, segment 2100 of FIGS. 21A and 21B may not hold any optical sensor in some instances.

The segment 2200 may be formed of any suitable materials, including any of those previously described herein for supports or any other suitable materials. Thus, in some embodiments the segment 2200 may be configured to conform to a subject, may be soft, padded, cushioned, stretchable, flexible, or have any other suitable material construction.

In some embodiments, the segment 2200 may include a foam cushion having holes formed therein. The holes may allow the optical sources and/or detectors of an optical sensor to protrude from the foam cushion and contact a subject. However, the thickness of the foam cushion may be selected such that the optical sources and/or detectors protrude by a relatively small amount, such that the cushion may serve to cushion the optical sensor against the subject, thus providing increased comfort.

The segment 2200 may have any suitable dimensions for supporting an optical sensor and conforming with an intended subject. For example, in the context in which the segment 2200 is to be configured in the manner shown for second segment 2012 of FIG. 20A (i.e., to engage with the front and sides of a subject's head), L4 may be between approximately 15 inches and approximately 35 inches, between approximately 20 inches and approximately 30 inches, may have any value within such ranges, may be approximately 24 inches, approximately 26 inches, or any other suitable value. The value of W1 may likewise having any suitable value for an intended manner of use. In some embodiments, W1 may be selected to be approximately the same width as an optical sensor held by the segment 2200. In some embodiments, W1 may be sufficiently narrow to allow access to the subject around the support, for example allowing access to the top of a subject's head as shown in FIGS. 20A and 20B. As non-limiting examples, W1 may be between approximately 1 inch and approximately 5 inches, may have any value within that range, may be approximately 3 inches, approximately 4 inches, or any other suitable value.

The segment 2200 may have various constructions. FIGS. 23A and 23B illustrate a more detailed non-limiting example of the segment 2200. FIG. 23A illustrates an inner surface of the segment 2300, i.e., the surface intended to face the subject when the segment 2300 is in engaged with the subject while FIG. 23B illustrates an outer surface of the segment 2300, i.e., the surface intended to face away from the subject when the segment 2300 is engaged with the subject.

As shown in FIG. 23A, the segment 2300 may be formed of multiple pieces, including a first piece 2302, a second piece 2304 (also shown in FIG. 24), and a third piece 2306. The first piece 2302 may be a unitary body to which the second piece 2304 and third piece 2306 may be connected, in some instances in a manner that allows the second piece 2304 and third piece 2306 to slide relative to the first piece 2302, as will be described further below.

One or more of the first piece 2302, second piece 2304, and third piece 2306 may include a plurality of fasteners (or couplers) 2308 for engaging with or mechanically coupling to an optical sensor, in some embodiments the coupling being detachable. In the non-limiting example shown, each of the first piece 2302, second piece 2304, and third piece 2306 includes four fasteners (or couplers) 2308. The fasteners 2308 may be elastic bands, hook and loop components, adhesive pads, or any other suitable type of fastener. In some embodiments, a pouch, pocket, or open-faced frame may be used as the fastener with an optical sensor being inserted into the pouch/pocket/frame. In some embodiments, the fasteners 2308 may be configured to engage the corners of an optical sensor such as optical sensor 200. For example, an optical sensor may be rectangular and each of the fasteners 2308 of the second piece 2304 may engage a respective corner. In some embodiments, it may be desirable for the fasteners to be easily engaged with and disengaged from the optical sensor. In this manner, the segment 2300 (and support more generally) may be removed from an optical sensor and discarded. A new segment 2300 may then be used with the optical sensor.

In some embodiments, in addition to the fasteners 2308, at least part of the inner surface of the first piece 2302, second piece 2304, and/or third piece 2306 may be configured to restrict motion of an optical sensor when the optical sensor is in place. For example, the inner surface of the first piece 2302, second piece 2304, and/or third piece 2306 may be textured, may be rough, or may have other surface features which minimize or prevent movement/motion of the optical sensor against the surface.

As described, the second piece 2304 and third piece 2306 may be coupled to the first piece 2302 in a manner that allows them to slide relative to each other. For example, the second piece 2304 may be coupled to the first piece 2302 by a ring 2310a, which may represent or define a coupling point for coupling the first piece 2302 and second piece 2304. A non-limiting example of such a ring is illustrated in FIG. 25. As shown, the ring 2310a may include a body 2502 and a hole 2504. The ring 2310a may be fixedly attached to the second piece 2304, as also shown in FIG. 24, thus defining a coupling point of the second piece. For example, the second piece 2304 may surround part of the body 2502. The first piece 2302 may pass through the hole 2504, such that the ring 2310a may slide along the length of the first piece 2302 (see, e.g., FIG. 23B) and be removed from the first piece 2302. Thus, the first piece 2302 and second piece 2304 may be separable from each other and separately replaced or discarded.

The third piece 2306 may be attached to the first piece 2302 by a ring 2310b. The construction and operation of ring 2310b may be substantially the same as that of ring 2310a. Thus, the ring 2310b may define a coupling point of the third piece 2306 for coupling to the first piece 2302. The first piece 2302 and third piece 2306 may be separable from each other and separately replaced or discarded.

The first piece 2302 may also be coupled to the second piece 2304 and third piece 2306 at coupling points represented by the respective ends 2322 and 2324 of the second piece 2304 and third piece 2306, i.e., the second piece 2304 may be said to have a coupling point represented by end 2322 and the third piece 2306 may be said to have a coupling point represented by end 2324. The location of these coupling points relative to the first piece 2302 may be used to adjust the sizing of the support and the placement/positioning of optical sensors held by the second piece 2304 and third piece 2306 relative to the subject (e.g., the placement of optical sensors proximate the sides of the subject's head).

The coupling points represented by ends 2322 and 2324 may be coupled to the first piece 2302 by respective fasteners 2312, which may be adjustable in some embodiments. The fasteners 2312 may hold the first piece 2302, second piece 2304, and third piece 2306 in a relatively fixed position with respect to each other. However, the fasteners may be adjustable in that the placement at which second piece 2304 and third piece 2306 are coupled to the first piece 2302 may be adjusted. As an example, the fasteners 2312 may each have a width W2, and the ends 2322 and 2324 (representing coupling points) may be coupled to the first piece 2302 anywhere across the widths W2. In this manner, the location of coupling may be adjusted, and thus the size of the support may be adjusted as well as the positioning of the second and third pieces 2304 and 2306, and any optical sensors they may hold, relative to the subject when the support is in place.

The fasteners 2312 may be any suitable type of fasteners, and in some embodiments may be adjustable fasteners. In some embodiments, the fasteners may be hook and loop fasteners. For example, the fasteners 2312 may include hook portions and the second piece 2304 and third piece 2306 may be formed of a material (e.g., a fabric or other suitable material) which engages with the hook portions. In some embodiments, the second piece 2304 and third piece 2306 are detachable from the fasteners 2312, to provide the adjustable nature described above.

The pieces illustrated in FIG. 23A may be assembled in any suitable manner. As a non-limiting example, the first piece 2302 may be slid through the hole 2504 of ring 2310a and the second piece 2304 detachably (and adjustably) fastened to the first piece 2302 with a respective fastener 2312. The first piece 2302 may be slid through the hole of ring 2310b and the third piece 2306 detachably (and adjustably) fastened to the first piece 2302 with a respective fastener 2312.

The second piece 2304 and third piece 2306 may further comprise respective openings (or holes) 2314. Such openings 2314 may allow for a strap or other connector from a different segment (e.g., from segment 2100) to engage the second piece 2304 and third piece 2306. For example, in a non-limiting embodiment, a first end of strap 2104 may pass through opening 2314 of second piece 2304 and the other end of strap 2104 may pass through opening 2314 of third piece 2306. The strap 2104 may then be folded such that the fasteners 2118a and 2118b connect back to the body 2102 of the segment 2100. An example of such a configuration is illustrated in connection with FIGS. 26-28.

Based on the foregoing, it should be appreciated that in some embodiments the segment 2100 and segment 2300 may be coupled together to form a loop or other closed contour. Specifically, in some non-limiting embodiments, the strap 2104 of segment 2100 may engaged the openings 2314 of second piece 2304 and third piece 2306 such that the segment 2100, second piece 2304, third piece 2306, and the portion of first piece 2302 between fasteners 2312 may form a loop. This loop may be fitted to a subject's head (or other region of interest). The size of the loop may be controlled, at least in part, by adjusting the strap 2104 and, in some embodiments, the straps 2106a and 2106b, which may be connected to the segment 2300.

It should be appreciated that merely engaging the strap 2104 with the second piece 2304 and third piece 2306 to form a loop does not necessarily tightly engage the ends 2303a and 2303b of the first piece 2302. Those ends 2303a and 2303b, which themselves may be considered straps anchored on the segment 2300 in some embodiments, may be used as tensioners or tighteners to adjust the tension (or fit or pressure or sizing) of the loop, as will be described further below. For example, pulling the ends 2303a and 2303b toward the front of the head may serve to tighten the support and increase the pressure of the optical sensors against the head (or subject more generally).

In some embodiments, the supports may include other features or mechanisms to control/adjust the pressure exerted by optical sensors against a subject. For example, compression elements (e.g., mechanical springs, inflatable chambers such as air bladders, or other compression elements) may be included as part of the supports. When included, such compression elements may provide an independent mechanism for adjusting the pressure of optical sensors against the subject.

As shown in FIG. 23B, in a non-limiting embodiment the first piece 2302 may have three portions 2318a-2318c, though not all embodiments are limited in this respect. The portions 2318a-2318c may represent different materials in some non-limiting embodiments. For example, portion 2318b may be a first material and portions 2318a and 2318c may be a second material. As a non-limiting example, the portion 2318b may be a cloth material, a cushioned material, or any other suitable material, and in some embodiments may be formed of substantially the same material(s) as second piece 2304 and third piece 2306. The portions 2318a and 2318c may be formed of a material exhibiting a higher capability for stretching, such as rubber, neoprene, or any other suitable material. In some embodiments, the portions 2318a and 2318b may function as tensioners and thus may be formed of a suitable material for stretching and applying tension to the support when engaged with a subject. For example, the portions 2318a and 2318b may be straps which, when pulled toward the front of the subject's head, tighten the support and therefore increase the pressure of the optical sensor held in contact with the subject.

As shown, the ends 2303a and 2303b may include respective fasteners 2320a and 2320b. The fasteners 2320a-2320b may serve to fasten the respective ends 2303a and 2303b of first piece 2302 to a desired point for providing a desired fit or level of tension to the support. As a non-limiting example, the end 2303a may be folded back over the ring 2310a such that the fastener 2320a may be engaged with the portion 2318b. For example, the fastener 2320a may form a hook and loop closure with the portion 2318b. Similarly, the end 2303b may be folded back over the ring 2310b such that the fastener 2320a may be engaged with the portion 2318b, for example by forming a hook and loop closure or other suitable fastening closure.

The fasteners 2320a and 2320b may be any suitable fasteners, as the various aspects described herein are not limited in this respect. For example, the fasteners 2320a and 2320b may be hook and loop components, clips, buckles, adhesive pads, or other fasteners, and in some embodiments may form detachable closures.

The first piece 2302 may optionally include an indicator 2316, which may be any type of indicator as previously described in connection with indicator 2114 or any other suitable indicator or any other suitable indicator. The indicator 2316 may be used to aid user alignment of the segment 2300 with a desired feature of a subject. For example, the indicator may be aligned by the user with the a subject's forehead to ensure that optical sensors held by the support are properly positioned with respect to the subject. Depending on the nature of the indicator 2316, it may or may not be visible on the inner surface of the segment 2300 and thus is shown with dashed lining in FIG. 23A.

As should be appreciated from the foregoing, supports according to one or more aspects of the present application may include multiple segments (or pieces). The segments may be connected in various manners. For example, first piece 2302, second piece 2304, and third piece 2306 may, in some embodiments, be considered to part of a single segment. Alternatively, as previously described, the second piece 2304 and third piece 2306 may be separated from the first piece 2302 (e.g., by sliding the first piece 2302 out of rings 2310a and 2310b), but may be coupled to segment 2100 by straps 2104, 2106a and 2106b. Thus, the second piece 2304, third piece 2306, and segment 2100 may, in some embodiments, be considered to form a single segment for coupling to a rear portion and side portions of a subject's head. That segment may, in some embodiments, be configured to hold one or more optical sensors (e.g., one being held by each of second piece 2304 and third piece 2306).

Considering such a configuration, an aspect of the present application provides a support having a first (rear) segment configured to couple to a rear portion of a subject's head and having two forward coupling points (e.g., the ends 2322 and 2324) and two rear coupling points (e.g., defined by rings 2310a and 2310b). The support may further include a second (front) segment having a center portion configured to adjustably couple to the forward coupling points of the first segment and having two ends configured to slidably (or otherwise variably) couple to the two rear coupling point of the first segment. The ends of the second segment may function as tensioners to adjust a tension of the support by actuating the slidable coupling to the first segment (e.g., by pulling the ends of the second segment forward away from the first segment).

In some embodiments, the second piece 2304 and third piece 2306 may be considered o each have multiple (e.g., two) coupling points. For example, the second piece 2304 may have coupling points defined by end 2322 and ring 2310a. The third piece 506 may have coupling points defined by end 2324 and ring 2310b. One coupling point for each may be used to adjust a sizing of the support and/or a positioning of an optical sensor relative to a subject. Another coupling point of each piece 2304 and 2306 may be used to adjust a tension of the support (e.g., by accommodating a tensioner).

According to an aspect of the present application, a support may comprise two straps. A first strap may be considered to engage with a rear portion and, optionally, sides of a subject's head. A second strap may be configured to engage with a front portion and, optionally, sides of the subject's head. The first and second straps may be couplable to each other via one or more first adjustable coupling points. One or more additional coupling points may serve as points via which to apply tension to the support. In some embodiments, the first adjustable coupling points may be configured to be positioned between optical sensors held by the support. For example, end 2322 when fastened is located between the optical sensors held by first piece 2302 and second piece 2304 and the end 2324 when fastened is located between the optical sensors held by first piece 2302 and third piece 2306. The additional coupling points may be located substantially on opposite ends of the optical sensors. For example, the rings 2310a and 2310b may be positioned substantially opposite the ends 2322 and 2324 and thus on opposite ends of the optical sensors held by the second piece 2304 and third piece 2306.

In some embodiments, a support comprising four pieces is provided. The support may include front, rear, and two side pieces. The side pieces may be coupled to the front and rear pieces in any suitable manner to form a substantially closed contour. Any one or more of the pieces may be configured to hold an optical sensor.

FIG. 26 shows an example of a support including first and second segments coupled together, absent a subject. As shown, the support 2600 includes previously described segment 2100 coupled to previously described segment 2300. The strap 2106 is fed through the openings 2314 and fastened on the segment 2100. The straps 2106a and 2106b of segment 2100 extend toward and are fastened to the segment 2300. It can be seen that the coupled segments 2100 and 2300 form a closed contour or loop.

FIG. 27 shows an example of two-piece support 2700 including segment 2300 and segment 2702 coupled together and mounted to head 2002. The segment 2702 may be similar to previously described segment 2100, and may include the strap 2104. As shown, the strap 2104 may pass through openings 2314 of segment 2300 and be fastened to the segment 2702, thus coupling the segment 2300 and segment 2702 together to form a substantially closed contour.

FIG. 28 illustrates a rear perspective view of a support comprising segment 2100 coupled to segment 2300 and mounted to head 2002. As shown, the support may support an optical sensor 2802 against the head 2002.

FIG. 29 illustrates a front perspective view of the support of FIG. 28. As shown, the first piece 2302 may support two optical sensors 2802. The end 2322 is represented with a dashed line to indicate it is beneath the surface of the support illustrated. Also, it should be noted that the support leaves the top of the head 2002 substantially open. For example, drainage points 2902 may be accessible to provide doctors the ability to insert medical instrumentation (e.g., drains or other instruments) and leave the instrumentation in place even when the support is engaged with the head 2002. In this manner, optical analysis of the head (or subject more generally) may be performed by the optical sensors 2802 while allowing for other procedures, evaluation, or treatments to be ongoing on the top of the head.

It should be appreciated that various manners of applying supports of the types described herein to a subject are possible, some of which have been previously described. As a non-limiting example, a manner of applying a support comprising segments 2100 and 2300 is now described. The method may begin by engaging at least one fastener or connector to form a loop at least partially defined by the segment 2100 and segment 2300. For example, strap 2104 of segment 2100 may be fed through the openings 2314 of second piece 2304 and third piece 2306 and the fasteners 2118a and 2118b fastened to the segment 2100.

The loop formed by segments 2100 and 2300 may then be placed about the subject's head such that the loop wraps substantially around a circumference of the subject's head. At least one tensioner may then be actuated to adjust the tension of the loop around the subject's head. For example, ends 2303a and 2303b, which may be positioned proximate opposed sides of the subject's head, may be pulled toward the front of the subject's head and fasteners 2320a and 2320b fastened to an outer surface of first piece 2302. Thus, a desired tension of the support around the subject's head may be achieved.

Next, straps 2106a and 2106b of segment 2100 may be fastened to an outer surface of segment 2300. For example, straps 2106a and 2106b may be fastened to the ends 2303a and 2303b. In this manner, lower portions 2108a and 2108b may be made to lie flush with the subject's head and provide additional tension/fit control.

In some embodiments, the support may be placed about the subject's head prior to forming a completed loop. For example, second piece 2304 and third piece 2306 may be coupled to the segment 2100 using the strap 2104. The second piece 2304 (or third piece 2306) may be coupled to the first piece 2302, for example with the fastener 2312. The support may then be placed about the subject's head and a completed loop then formed by coupling the remaining one of second piece 2304 and third piece 2306 to the first piece 2302 with the fastener 2312. The support may then be tightened. For example, one or both of the ends 2303a and 2303b may be free at this stage, and may be fitted through respective rings 2310a and 2310b, pulled tight, and, using fasteners 2320a and 2320b, fastened to an outer surface of first piece 2302. According to this approach, the support may be positioned about the subject's head without disturbing objects (e.g., drains) on the subject's head.

It should be appreciated from the foregoing that in some embodiments supports may include distinct mechanisms for forming a support loop and for tightening the loop. For example, a loop may be formed as described with strap 2104, which in forming the loop may provide some control over size/tension of the support. However, ends 2303a and 2303b (or other suitable tensioners) may act independently to adjust the sizing/tension of the loop once formed.

As previously described, in some scenarios it may be desirable to replace a support of the types described herein while reusing the optical sensor(s) supported by the support. Thus, the process described above for engaging the support may be repeated. For example, after the support has been fitted to the subject and when it is desired to replace the support, the support may be removed by decoupling segments 2100 and 2300. The optical sensor(s) may be removed from the segment 2300 and segment 2100 and/or 2300 may be discarded. New segments 2100 and 2300 may be obtained, and the optical sensor(s) coupled to the segment 2300. The segments 2100 and 2300 may then be coupled together and fitted to the subject (the original subject or a new subject) in the manner previously described. In this manner, the support may be replaced.

Although various examples of supports have been described herein, it should be appreciated that alternatives falling within one or more aspects of the present application are possible. For example, one or more additional straps may be added to the supports described herein. As a non-limiting example, a chin strap may be included with the supports described herein, for example to prevent unwanted movement of the support toward the top of the subject's head. Alternatively or additionally, an overhead strap may be included with the supports described herein, configured to pass over a top portion of the subject's head. Such a strap may prevent unwanted downward movement of the support. Such a strap may also be used to apply additional pressure inward on the support (i.e., toward the subject's head).

Moreover, it should be appreciated that supports of the types described herein may, in some embodiments, be substantially reversed. For example, rather than a configuration in which a support segment is provided to couple to the front of a subject's head and for which tension is applied by pulling straps toward the front of the subject's head, the tensioning may be configured to be pulled toward the rear of the subject's head (e.g., the sizing and tensioning functions may be substantially reversed compared to the orientations described in some of the preceding examples). Other configurations are also possible.

Various benefits may be provided by one or more aspects of the present application. Following is a description of some benefits which may be achieved from implementing one or more aspects. However, it should be appreciated that not all aspects necessarily provide all listed benefits, and that benefits other than those listed may be provided. Thus, the benefits described herein are non-limiting examples.

Aspects of the present application provide for easily applied and removed supports for optical sensors. The supports may be formed of materials that are comfortable to the wearer, safe in a medical environment, and relatively inexpensive. The supports may easily engage with and disengage from an optical sensor, such that the supports may be disposable. The supports may provide multiple mechanisms for adjusting the sizing/fit of the support and the pressure of the optical sensor against the subject. Thus, accurate and comfortable fit may be achieved.

Aspects of the present application relate to liners for optical tomography sensors and related apparatus and methods. As previously described, an optical sensor (e.g., sensor 200) may be positioned to contact a subject. Such positioning may be beneficial and/or necessary in some embodiments to ensure accurate operation of the sensor. However, direct contact between the optical components (e.g., optical sources and optical detectors) and the subject may be undesirable for various reasons, and thus aspects of the present application provide for a liner to be placed on the optical sensor.

Direct contact between an optical sensor and a subject (e.g., a patient) may be harmful to the subject and/or the sensor. For example, if the optical sensor is to be used on multiple subjects, then direct contact of the optical sensor with multiple subjects may represent a bio-contamination hazard, a re- or cross-infection hazard, and more generally compromise hygienic safety. Cleaning the optical sensor itself may be difficult if it was to become soiled. If direct contact is made between the optical sensor and the subject, the optical sensor itself may be damaged, for example by getting scratched or otherwise modified in a manner that could be detrimental to the sensor operation.

Accordingly, aspects of the present application provide liners for use with optical sensors of the types that may be used in optical tomography systems, such as sensor 200 and the system of FIG. 1. The liners may serve to protect the optical sensor as well as the subject when direct contact is to be made between the subject and the optical sensor. In some embodiments, the liners and/or optical sensors themselves may include features to increase comfort of the subject when contacted by the optical sensor, such as soft portions providing cushioning functionality. The liner may be disposable, allowing for re-use of the optical sensor with a new liner. In this manner, bio-contamination may be minimized and the likelihood of needing to replace the optical sensor, which may be a relatively complex and expensive piece of equipment, may also be minimized.

According to an aspect of the present application, a liner for an optical sensor of the type that may be used in an optical tomography system (e.g., system 100 of FIG. 1) is provided. The liner may be disposable in some embodiments, and thus may be readily applied to and removed from the optical sensor. The liner may be constructed to have desirable optical properties, such having a portion that is substantially opaque (e.g., to wavelengths implemented by the optical sensor, environmental optical signals such as ambient sunlight, light bulbs, etc.) and a portion that is substantially transparent to wavelengths implemented by the optical sensor.

A liner according to an aspect of the present application may be implemented with various types of optical sensors having various configurations, a non-limiting example of which is the optical sensor 200. Suitable liners for use with such an optical sensor are shown and described in connection with FIGS. 30A-30C. However, it should be appreciated that configurations of liners other than those shown in FIGS. 30A-30C may be implemented depending on the configuration of the optical sensor.

FIG. 30A illustrates a top perspective view of a liner 3000 (which may also be referred to herein as a cover or protector) which may serve as a liner or cover for the optical sensor 200 of FIG. 2, according to a non-limiting embodiment of the present application. The liner 3000 includes a flexible sheet 3002 with a plurality of indentations 3004 formed therein. The indentations 3004 may also be considered protrusions depending on perspective, and may be hollow. In the embodiment illustrated, the liner 3000 includes one indentation 3004 for each of the optical components (optical sources and optical detectors) of the optical sensor 200.

The liner 3000 may be configured to align with and engage with (or couple to, mate to, or other similar terminology) the optical sensor 200 of FIG. 2. For example, the indentations 3004 of the liner 3000 may be arranged in the same manner (or substantially the same manner) as the optical source and optical detectors of the optical sensor 200 and thus in some embodiments may be arranged in an array. Thus, the liner 3000 may be aligned with the optical sensor 200 by aligning the indentations 3004 with the optical sources 202 and optical detectors 204. The liner 3000 may then be mechanically engaged with the optical sensor 200 in any suitable manner, for example by press-fitting, by hand or machine, or in any other suitable manner. The engagement may be detachable (or removable or decouplable), i.e., the liner may be disengaged from the optical sensor.

The liner 3000 may optionally include a tab 3006 or other suitable feature for facilitating removal of the liner 3000 from an optical sensor. For example, when it is desired to remove the liner 3000 from an optical sensor (e.g., when switching between a first subject and a second subject), a user may grasp the tab 3006 and pull the liner 3000 off the optical sensor 200. While the liner 3000 is illustrated as including a tab 3006, it should be appreciated that other structures (e.g., other than a tab) may alternatively or additionally be provided to facilitate removal, and more generally handling, of the liner 3000.

The liner 3000 may have any suitable dimensions. In some embodiments, the liner 3000 may have a length L5 in the y-direction in FIG. 30A approximately equal to the length of the optical sensor 200 and thus having any length previously described in connection with the optical sensor 200 or any other suitable length, and a width W3 in the x-direction in FIG. 30A approximately equal to or less than the width of the optical sensor and thus having any width previously described in connection with the optical sensor 200 or any other suitable width.

The liner 3000 may have a thickness T1 that is relatively small compared to L5 and W3 in some embodiments. The thickness T1 may be the thickness of substantially all of the liner 3000, including the indentations 3004 as well as the portions of the flexible sheet 3002 between the indentations 3004 though not all embodiments are limited in this respect. In some embodiments, the thickness T1 may be uniform for the entire flexible sheet, whereas in other embodiments the flexible sheet may have a varying thickness, and T1 may represent the maximum thickness or an average thickness. The thickness T1 (whether a maximum, average, or uniform value) may be, for example, less than approximately 20 mm, less than approximately 10 mm, less than approximately 5 mm, less than approximately 3 mm, less than approximately 2 mm, between approximately 0.5 mm and approximately 2 mm, or any other suitable value. As previously described, the liner may be flexible in some embodiments, and choosing a small thickness T1 may facilitate flexing of the liner. Moreover, since the liner 3000 may overlie the optical sources 202 and optical detectors 204 it may be desirable for the liner 3000 to have a small thickness to facilitate positioning of the optical sources 202 and/or optical detectors 204 close to a subject (e.g., a patient's head).

In some embodiments, the liner may be substantially as large as or larger than an optical sensor. For example, the liner may cover not only the optical sources and optical detectors of an optical sensor, but any electronics (e.g., circuitry modules 208a-208c). In some embodiments, the liner may substantially encase the optical sensor though allowing for a cable or other connector between the optical sensor and external components. For example, in some embodiments the liner may be a pouch or bag into which the optical sensor may be placed.

The indentations 3004 may be sized to accommodate the optical sources 202 and optical detectors 204 therein. For example, the indentations 3004 may have a width (e.g., a diameter or other width) and height, illustrated and described below in connection with FIG. 30C, suitable to fit an optical source and/or optical detectors therein. In some embodiments, the internal dimensions of the indentations may be substantially the same as the external dimensions of the optical sources 202 and/or optical detectors 204 such that the optical sources and/or optical detectors may fit into the indentations 3004 with a negligible gap or no gap between the optical sources/detectors and the indentations. Such an arrangement may be optically beneficial, since a gap (e.g., filled with air) between the optical sources/detectors and the liner may impact the optical performance of the optical sensor. Furthermore, such a relative sizing of the optical sources/detectors and the liner 3000 may facilitate formation of a good friction fit between the two, thus minimizing or eliminating the need in some embodiments for any adhesive or additional fastening mechanism to be used to couple the liner 3000 to the optical sensor 200.

As described, in some embodiments a liner may be sized and applied to an optical sensor to prevent an air gap between the optical components (e.g., optical sources/detectors) and the liner. In addition to suitable sizing of the liner, the liner may include small openings/holes suitable positioned (e.g., on a tip of the indentations 3004) to allow air to escape. Alternatively, a channel (or more than one channel) may be formed on an inner surface of the liner to allow air to move from over the optical source/detector toward a base part of the liner.

In an alternative embodiment, the indentations 3004 may be replaced by sections of stretchable material (e.g., polyurethane). For example, the liner may be formed of two materials, one being relatively non-stretchable and a plurality of stretchable portions arranged in substantially the manner of indentations 3004. The liner may then be placed over an optical sensor and the stretchable portions (e.g., formed of a stretchable film) may stretch to conform to the optical sources and optical detectors of the optical sensor, thus assuming a shape much like that of the indentations 3004. In some such embodiments, the stretchable portions may be optically clear (as described further below) and the remainder of the liner may be optically opaque.

The liner 3000 may be formed of any suitable material, which in some embodiments may be a biocompatible material. In some embodiments, the material may be non-allergenic. As previously described, the liner 3000 may be flexible in some embodiments, and thus may be formed of a flexible material, such as a rubber. The liner may be soft or pliable, and thus in some embodiments may operate as a soft cover for an optical sensor. The material may provide desired optical properties for the liner 3000. For example, the indentations 3004 or a portion thereof (e.g., the tips of the indentations) may be formed of a material that is optically transparent to the wavelengths implemented by the optical source 202 and optical detectors 204. The remainder of the flexible sheet 3002 may be formed of a material that is optically opaque to the wavelengths implemented by the optical sources 202 and optical detectors 204, i.e., the portion of the liner between the indentations may be optically opaque. In this manner, undesirable tunneling or channeling of optical signals from an optical source through the support structure 206 to an optical detector of the optical sensor 200 may be avoided. Thus, according to a non-limiting embodiment, the indentations 3004 may be formed of optically clear material such as NuSil-6033, and the remainder of flexible sheet 3002 may be formed of opaque material such as NuSil MED-6033, with Silcopas 220 black.

The liner 3000 may be formed of a material providing desired mechanical properties. For example, as previously described, the liner 3000 may be intended to be applied to and removed from an optical sensor (e.g., optical sensor 200) and thus it may be desired for the liner 3000 to be formed of a material that is capable of stretching and resisting tearing. In some embodiments, the flexible sheet 3002 may be formed of a material having an elongation of at least 150%, between approximately 100% and approximately 900%, any value in between, or any other suitable value. In some embodiments, the material may have a tear strength of approximately 80 pounds per inch (ppi), between approximately 30 ppi and approximately 100 ppi, any value in between, or any other suitable value. In some embodiments, the material may have a durometer of 50 A, between 10 A and 70 A, any value in between, or any other suitable value. In some embodiments, the liner may be formed of a material which is capable of being cleaned (e.g., by wiping).

FIG. 30B illustrates an alternative non-limiting embodiment of a liner 3020 having a flexible sheet 3022 with the indentations 3004, and having a length L6, a width W4, and a thickness T2. The liner 3020 may be used in connection with an optical sensor such as optical sensor 200 of FIG. 2. As shown, the liner 3020 may have a pre-curvature to it, for example around one or more axes, which may be used, for example, if the optical sensor has a curvature to it. Nonetheless, the liner 3020 may be flexible as with the previously described liner 3000 and may be formed of the same materials as those previously described in connection with liner 3000 or any other suitable materials. The values of L6, W4, and T2 may take any of the values previously described in connection with L5, W3, and T1, respectively, or any other suitable values.

FIG. 30C illustrates a side view of a portion of liner 3000 of FIG. 30A. As shown, each of the indentations 3004 may include a first portion 3008 and a second portion 3010. The first portion and second portion may exhibit differing optical properties. For example, the first portion 3008 may be substantially opaque to wavelengths implemented by an optical source 202 and optical detector 204 around with the indentation 3004 is to be fitted. The second portion 3010 may be substantially optically transparent (or transmissive) to such wavelengths. In this manner, the first portion 3008 may minimize or prevent undesired cross-talk between optical sources 202 and optical detectors 204, while the second portion 3010 may permit the desired operation of the optical sources 202 and optical detectors 204.

The first portion 3008 may be, in some embodiments, considered the base or bottom portion of a columnar structure of the indentation, and the second portion may be considered the top portion or cover portion of the indentation. The second portion 3010 may also be referred to as a tip (e.g., an optical tip, optically transparent tip, or other similar terminology). As a non-limiting example, the second portions 3010 may be formed of NuSil MED-6033 or thin polyurethane, which may be optically clear. The first portions 3008 may be formed of NuSil MED-6033, with Silcopas 220 black, or a black polyurethane sheet. In some embodiments, the second portion 3010 may not be included with the liner, i.e., the indentations 3004 may be holes where the second portion 3010 is replaced by an opening in the liner.

The first portion 3008 and second portion 3010 may have any suitable dimensions. In some embodiments, the first portion 3008 may have a height H9 and the second portion 3010 may have a height H10. The height H10 may be selected to be just large enough to provide a desired emission/reception angle for an optical source/optical detector, respectively, to be fitted inside the indentation 3004, in some embodiments. In some embodiments, the height of H10 may be between approximately 1 mm and approximately 6 mm, between approximately 2 mm and approximately 4 mm, approximately 1 mm, approximately 1.5 mm, approximately 2.5 mm, less than 5 mm, less than approximately 3 mm, less than 2 mm, any value between 1 mm and 5 mm, or any other suitable value. The height H9 may then represent the remaining height of the indentation 3004, and may assume any suitable values, such between approximately 2 mm and 20 mm, between approximately 2 mm and 10 mm, between approximately 3 mm and 7 mm (e.g., 4 mm, 5 mm, or 6 mm), any value within such ranges, or any other suitable height.

The indentations 3004 may have a width D4 (e.g., a diameter or other width) of any suitable value. The width may represent the inner width of the indentation or an outer width. The walls of the indentations may be thin (e.g., having any of the thicknesses previously described in connection with T1 or any other suitable thickness, though in some embodiments it may be desirable for the walls of the indentations to be thinner than T1, such as on the order of 1 mm). As non-limiting example, D4 may be between approximately 3 mm and approximately 10 mm, between approximately 4 mm and approximately 7 mm, approximately 4.5 mm, approximately 5 mm, any value in those ranges, or any other suitable width.

As a non-limiting example, the liner 3000 illustrated in FIG. 30C may have a thickness T1 less than approximately 5 mm, indentations 3004 having a height H9+H10 less than approximately 10 mm, and a width D4 less than approximately 5 mm. The liner 3000 may be flexible and configured with an array of indentations 3004 to align and engage with (or couple to) an array of optical sources and detectors.

As previously described in connection with FIG. 30A, the indentations 3004 may have dimensions (e.g., D4, H9, and H10) selected such that the dimensions substantially equal the outer dimensions of the optical sources 202 and/or optical detectors 204 which are to fit inside the indentations 3004. In this manner, a tight fit (e.g., a friction fit) may be achieved when the liner 300 is placed on (or engaged with) the optical sensor 200.

FIG. 30C also shows that the backside 3012 of the liner 3000 may be substantially flat (other than the indentations formed therein). The backside 3012 may have a surface contour selected in dependence on a surface contour of the optical sensor with which the liner 3000 is to be engaged. For example, if the optical sensor has a substantially smooth upper surface, the backside 3012 of the liner 3000 may be made substantially smooth to facilitate proper (detachable) engagement of the liner with the optical sensor. Thus, the surface contour of backside 3012 may take various suitable forms depending on the types of optical sensors with which the liner 3000 is to be used.

Liners of the types described herein may be fabricated in any suitable manner. According to a non-limiting embodiment, a liner may be molded. In some embodiments, a multiple step (e.g., two-step) molding processing may be used. For example, considering the liner 3000 illustrated in FIG. 30A, a two-step (or two-shot) molding process may involve molding the second portion 3010 in one molding step and the remainder of the liner in a separate molding step (in that order or in the reverse order). The second portion 3010 may be referred to a molded tip when formed by a molding process.

FIG. 31A illustrates an example of a device 3100 including the optical sensor 200 with the liner 3000 in place on the optical sensor 200. As shown, the indentations align with and engage mechanically with the optical sources 202 and optical detectors 204. The engagement (or coupling) may be detachable, such that the liner may also be disengaged (or decoupled) from the optical sensor.

FIG. 31B is an inset of FIG. 31A (representing portion 3101) and illustrates a cross-sectional view of the configuration of the liner 3000 with respect to a single optical detector 204. In particular, it can be seen that the optical detector 204 fits within the indentation 3004. For ease of illustration, the first portion 3008 and second portion 3010 of the indentation 3004 are not illustrated as distinct. In some embodiments, such as that shown in FIG. 31A, the optical detector may fit securely (or snugly) within indentation 3004. For example, intersection surface 3102 may represent the inner surface of the indentation 3004 and the outer surface of the optical detector 204, and as shown those two surfaces may be substantially flush with each other over substantially all of the outer surface of the optical detector. In this manner, a friction fit may be formed between the liner 3000 and the optical sensor 200, such that the liner 3000 may remain in place during normal operation (e.g., when placed against a subject).

As can also be seen from the inset of FIG. 31B, the indentation 3004 of the liner 3000 may be positioned such that when the optical sensor 200 is placed in contact with a subject (e.g., a patient), the liner 3000 is the structure making direct contact with the subject and not the optical sensor. In this manner, bio-contamination and damage to the optical sensor 200 may be minimized or avoided entirely.

As previously described, liners of the types described herein (e.g., liners 3000 and 3020 of FIGS. 30A and 30B, respectively), may be used as disposable or replaceable components. Optical sensors (e.g., optical sensor 200) may be relatively expensive and complex devices, and it may be desired to reuse them with multiple subjects. However, liners such as those described herein may be relatively inexpensive and therefore may be readily used and disposed of each time an optical sensor is used on a new subject or, in some instances, multiple times during use on the same subject. Thus, according to an aspect of the present application, a manner for applying and removing a liner of the types described herein may be provided.

It may be desirable to make application and removal of a liner from an optical sensor a relatively easy process, so that users can perform the operation without requiring significant time and without risking damage to the liner or the optical sensor. According to an aspect of the present application, an applicator device may be provided to facilitate applying a liner to an optical sensor. The applicator device may be handheld in some embodiments. A non-limiting example is illustrated in FIGS. 32A and 32B.

FIGS. 32A and 32B illustrate a top perspective view and bottom perspective view, respectively, of a device 3200 which may be used for applying a liner of the types described herein (e.g., liners 3000 and 3020) to an optical sensor, according to a non-limiting embodiment. As shown in FIG. 32A, the device 3200 may be a support structure having an upper surface 3202 and openings 3204 formed therein. The openings may be indentations, holes, or any other features suitable for accommodating the indentations of a liner (e.g., indentations 3004 of liner 3000). The openings 3204 may thus be arranged in substantially the same manner (i.e., having the same layout) as the indentations of a liner to be applied with the device 3200. FIG. 32B illustrates the back surface 3206 of the device 3200.

The upper surface 3204 may be formed to engage suitably with a flexible sheet (e.g., flexible sheet 3002) of a liner such that when the device 3200 is pressed onto an optical sensor the upper surface 3202 may force the liner onto the optical sensor. A non-limiting example of such operation will be described further below in connection with FIGS. 34A and 34B.

The device 3200 may be formed of any suitable material. In some embodiments, the device 3200 may be rigid (or substantially rigid) such that it may withstand pressure and be used to force a liner into place on an optical sensor when pushed. Thus, plastic, metal, or other suitable rigid material may be used to form the device 3200.

The device 3200 may have any suitable dimensions. For example, the support structure may have a length L7, a width W5, and a thickness T3. The length L7 may be substantially the same as the length of a liner to be applied with the device 3200, and thus may have any of the values previously described for the length of liners or any other suitable value, such as being less than approximately six inches, less than approximately 5 inches, or any other suitable value. The width W5 may be substantially the same as the width of a liner to be applied with the device 3200, and thus may have any of the values previously described for the width of liners or any other suitable value, such as less than approximately four inches, less than approximately three inches, or any other suitable value. The thickness T3 may be suitable to provide the device 3200 with sufficient rigidity and may, in some embodiments, be at least as large as or greater than the height of the indentions/protrusions of a liner to be applied by device 3200, such that the openings 3204 may have sufficient dimensions to accommodate the indentations/protrusions of the liner. As non-limiting examples, the thickness T3 may be between approximately ¼inch and 2 inches.

The openings 3204 may have a width D5 (e.g., a diameter or other width) of any suitable value to accommodate the indentations of a liner to be applied by the device 3200. In some embodiments, the width D5 may be sufficiently larger than the width of the indentations of a liner such that indentations may fit loosely within the openings 3204, i.e., the openings 3204 of the device 3200 may be wider than the indentations of a liner. In this manner, after the liner is applied to the optical sensor 200, an example of which is shown in connection with FIGS. 34A and 34B, the device 3200 may be removed without removing the liner from the optical sensor. As non-limiting example, D5 may be between approximately 3 mm and approximately 15 mm, between approximately 4 mm and approximately 10 mm, approximately 4.5 mm, approximately 5 mm, any value in those ranges, or any other suitable width.

The openings 3204 may have any suitable depth to accommodate the indentations of a liner. As can be seen from FIGS. 32A and 32B, in some embodiments the openings 3204 may be holes passing entirely through the device 3200. Thus, the openings 3204 may have a depth assuming any value previously described in connection with the thickness T3 or any other suitable value. It should be appreciated that the openings 3204 need not be holes in all embodiments, but rather may be indentations or other suitable features.

FIG. 33 illustrates a top perspective view of a liner engaged with the device 3200 of FIGS. 32A and 32B. The illustrated liner 3300 may be of the types previously described herein (e.g., liner 3000 or liner 3020), or any other suitable liner. As shown, the liner 3300 may be aligned with the device 3200 such that the indentations of the liner project into the openings 3204 of the device 3200.

FIGS. 34A and 34B illustrate a manner of using an applicator device 3400 to apply the liner 3000 to the optical sensor 200. The applicator device 3400 may be the same as the device 3200, or any other suitable applicator device. For purposes of simplicity, not all details of the applicator device 3400 and liner 3000 are shown.

As shown in FIG. 34A, the liner 3000 may be engaged with the applicator device 3400, for example in the manner previously shown and described in connection with FIG. 33. Thus, the indentations of the liner 3000 may (loosely) engage with the openings of the applicator device 3400 (not shown), for example by projecting into the openings of the applicator device 3400. The applicator device 3400 may then be aligned with the optical sensor 200 such that the indentations of the liner 3000 align with the optical sources 202 and optical detectors 204 of the optical sensor 200. The applicator device 3400 may then be moved toward the optical sensor 200 in the direction of the arrows to press the liner 3000 into a mechanically engaged state with the optical sensor 200. This procedure may be performed by hand or in any other suitable manner.

A force may be applied to the applicator device 3400 to ensure a good fit between the liner 3000 and the optical sensor. For example, a force may be applied to engage the liner 3000 and optical sensor 200 such that no gap exists between the two, including no air gap. As previously described, for example in connection with FIGS. 31A and 31B, minimizing any air gap between the liner 3000 and the optical sensor 200 may ensure proper optical operation of the optical sensor 200.

As shown in FIG. 34B, the applicator device 3400 may then be removed in the direction of the arrows, for example by lifting the applicator device 3400 by hand or in any other suitable manner. The liner 3000 may remain in place on the optical sensor 200 as shown. For instance, because of the relative sizing of the optical sensor 200, liner 3000, and applicator device 3400, the liner may engage more tightly with the optical sensor 200 than with the applicator device 3400.

As previously described, a liner (e.g., liner 3000 or 3020) may be removable (or detachable, or decouplable) from an optical sensor. Removal may be performed in any suitable manner. For example, referring to FIG. 34B, a user may grasp the tab 3006 of the liner 3000 and pull the liner off the optical sensor. In such embodiments, the liner may be peelable (capable of being peeled). The liner 3000 may then optionally be disposed of and a new liner put in place. Other manners of removing the liner are also possible.

While FIGS. 34A and 34B illustrate an embodiment in which a liner may be friction fit to an optical sensor, other engaging mechanisms may be used. For example, adhesives, straps, pins, hook and loop fasteners, or other techniques may be used to engage a liner with an optical sensor. Thus, the various embodiments described herein are not limited to friction fit engagements.

According to an aspect of the present application, a structure may be provided for controlling how an optical sensor makes contact with a subject. For example, considering the optical sensor 200, it can be seen that the optical sources 202 and optical detectors 204 may protrude above the support structure 206 and thus may act as points which contact the subject. Depending on the nature of the subject, the material used to form the optical sources 202 and optical detectors 204, and the pressure applied in coupling the optical sensor to the subject, such contact may be uncomfortable or damaging in some scenarios. For example, applying the optical sensor 200 to a subject's head may result in discomfort and/or leave a pattern of indentations in the subject's head from the optical sources 202 and optical detectors 204. According to an aspect of the present application, a structure may be provided to minimize discomfort.

FIG. 35 illustrates a structure which may be used to control how an optical sensor makes contact with a subject. The structure 3500 may be a pad that can be positioned on top of the optical sensor 200. For example, as shown, the structure 3500 may include a substrate 3502 having a plurality of holes 3504 formed therein. The holes 3504 may be arranged in a pattern to align with the optical sources 202 and optical detectors 204 of an optical sensor. Thus, the structure 3500 may be placed on top of the optical sensor 200 such that the optical sources 202 and optical detectors project into, and in some embodiments, all the way through, the holes 3504. A non-limiting example of such a configuration is described further below in connection with FIG. 36.

The structure 3500 may be formed of any suitable material. In some embodiments, the substrate 3502 may be formed of a soft or cushioning material and/or a compressible material, such as foam, rubber, or other soft material. In some embodiments, the structure 3502 may be formed of multiple layers. For example, a first layer may be formed of rubber and a second layer may be formed of foam. The first layer may be configured to contact an optical sensor and thus may be formed of a material that will resist moving relative to the optical sensor when the structure 3500 is mechanically engaged with (or coupled with) the optical sensor. The substrate 3502 may be formed of a material that is optically opaque in some embodiments, for example to prevent cross-talk between optical sources and optical detectors of the optical sensor.

The structure 3500 may have any suitable dimensions, including a length L8, a width W6, and a thickness T4. The length L8 may be substantially the same as the length of the optical sensor (or liner) to which the structure 3500 is to be applied, and thus may have any of the values previously described for example in connection with L5, or any other suitable. The width W6 may be substantially the same as the width of an optical sensor (or a liner) to which the structure 3500 is to be applied, and thus may have any of the values previously described, for example, in connection with W3. The thickness T4 may be selected to provide a desired relative positioning of the upper surface of the substrate 3502 and the tips of the optical sources 202 and optical detectors 204. For example, the thickness T4 may be between approximately 2 mm and 25 mm, between approximately 2 mm and 15 mm, between approximately 3 mm and 10 mm (e.g., 4 mm, 5 mm, or 6 mm), any value within such ranges, or any other suitable value.

The holes 3504 may have any suitable widths D6, which in some embodiments may be a diameter. As previously described, the holes may be sized suitably to allow the optical sources and/or optical detectors of an optical sensor to project through. Thus, the width D6 may be larger than the width of an optical source or optical detector. In some embodiments, the structure 3500 may be intended to fit over an optical sensor when a liner (e.g., liner 3000) is in place, and thus the holes 3504 may have widths D6 sufficiently large to accommodate the optical sources 202 and optical detectors 204 with the additional thickness of the liner. As non-limiting examples, the width D6 may be between approximately 3 mm and approximately 10 mm, between approximately 4 mm and approximately 7 mm, any value in those ranges, approximately 4 mm, approximately 5 mm, or any other suitable width.

It should be appreciated that the holes 3504 may have any suitable shape to accommodate optical sources and optical detectors. The circular shape illustrated is a non-limiting example. Alternative examples include rectangular holes, square holes, triangular holes, or any other suitable shape(s).

FIG. 36 shows a cross-sectional view of a portion of a device 3600 including the optical sensor 200 with the structure 3500 disposed thereon. As shown, the upper surface 3506 of the structure 3500 may be below the highest point (or maximum height) of the optical sources 202 (or other optical component, such as an optical detector) by a distance H11. H11 may have any suitable value, and the value may be selected depending on the intended use of the device 3600. For example, if the device 3600 is to be placed in contact with a subject's head, the value of H11 may be selected in dependence on the amount of hair the subject has. For example, H11 may be selected to be larger when the subject has more hair and smaller when the subject has less hair (e.g., being bald). As non-limiting examples, H11 may be between zero mm and 3 mm, less than 2 mm, less than 1 mm, any value within such ranges, approximately zero mm, or any other suitable value. Moreover, in some embodiments it may be desirable for the upper surface 3506 of the structure 3500 to be above the highest point of the optical sources 202 (i.e., for H11 in FIG. 36 to have a negative value).

In some embodiments, a structure such as structure 3500 may be configured to overlie a liner of the types described herein. For example, a liner (e.g., liner 3000 or 3020) may be applied to an optical sensor, and a structure (e.g., structure 3500) acting as a cushion may be placed over the liner. However, not all embodiments are limited in this manner.

In some embodiments, a structure such as structure 3500 may be considered a spacer, pad, cushion, or may be referred to by other similar terminology.

Various benefits may be provided by one or more aspects of the present application. Following is a description of some benefits which may be achieved from implementing one or more aspects. However, it should be appreciated that not all aspects necessarily provide all listed benefits, and that benefits other than those listed may be provided. Thus, the benefits described herein are non-limiting examples.

Aspects of the present application provide for easily applied and removed liners for optical sensors. The liners may minimize or eliminate bio-contamination and may protect the optical sensor itself. The liners may be relatively inexpensive and disposable and may minimize or obviate the need (and therefore the associated cost and effort) of cleaning an optical sensor. The liners may also increase the comfort of subjects (e.g., patients) to which the optical sensors may be coupled, for example by providing a relatively soft surface to make contact with the subject. In some embodiments, the liners may function as a thermal (e.g., heat) barrier between a subject and an optical sensor. For example, the liners may be formed of a thermally insulating material.

Optical tomography sensors and related apparatus and methods have been described. The present application covers the combination of all that is described herein. For example, the aspects described herein may be used individually, all together, or in any combination of two or more, as the present application is not limited in this respect.

Some non-limiting examples of the manner in which the aspects described herein may be combined are now described, though it should be appreciated that other aspects and embodiments may also be combined. As a first non-limiting example, the optical sensors (e.g., optical sensor 200 of FIG. 2A) may utilize any of the types of optical components described herein (e.g., the optical sources and optical detectors of FIGS. 15A-15D, 16A-16C, 17A-17D, 18 and 19). As a further example, the optical sources and optical detectors shown in FIGS. 3A-3C may be any of the types of optical sources and optical detectors described herein (e.g., those of FIGS. 15A-15D, 16A-16C, 17A-17D, 18, and 19).

Moreover, the optical components described herein may be operated such that different optical components emit different pluralities of center wavelengths, as described herein. For example, a first optical component of the type illustrated in FIGS. 16A and 16B may emit a first plurality of center wavelengths (e.g., four center wavelengths, with a respective center wavelength being emitted by each of the four optically active elements 1602) while a second optical component of the type illustrated in FIGS. 16A and 16B may emit a second plurality of center wavelengths (e.g., four different center wavelengths than the four center wavelengths emitted by the first optical component), the first and second optical components representing different optical sources of the optical sensor 200. Such operation may be achieved, for example, by providing the optical sources with multiple optically active emitting elements (e.g., optically active elements 1602). For example, an optical component of the type illustrated in FIG. 16A may include two, three, four, five, six, seven, eight, or any other suitable number of optically active emitting elements (e.g., LEDs) to emit the corresponding number of center wavelengths (e.g., a first plurality of wavelengths as described).

Thus, as a non-limiting example, an optical sensor of the types described herein may utilize optical sources and detectors of the types described herein, which may be operated in accordance with one or more aspects in which different optical sources emit different pluralities of center wavelengths).

As another example, it has been described that drive circuitry of an optical sensor may control operation of one or more optical sources of an optical sensor. For example, as described previously, drive circuitry may control the ON/OFF state of the optical sources (and therefore the duration of the optical signals emitted by the optical sources), the frequency modulation of the optical sources and/or the emission intensity and power of the optical sources (e.g., by controlling the current to the optical sources) of an optical sensor. Such control may be wavelength specific, meaning that the drive circuitry may control the described features (e.g., ON/OFF state, frequency modulation and/or emission intensity and power) of different wavelengths differently. Thus, for example, optical sensors of the type described herein may be operated such that different wavelengths of a first and/or second plurality of wavelengths as described herein may be independently controlled with the previously described drive circuitry.

As another non-limiting example, the supports described herein may be used to hold optical sensors of the types described herein. For instance, one optical sensor 200 may be held by each of the first piece 2302, second piece 2304, and third piece 2306 of the support of FIG. 23A. In some non-limiting embodiments, the fasteners 2308 of FIG. 23A engage the corners of the optical sensor 200 (e.g., one fastener 2308 may engage each of the corners by circuitry modules 208a and 208c of the optical sensor as well as the rounded corners opposite circuitry modules 208a and 208c). Other manners of coupling an optical sensor 200 to the fasteners 2308 are also possible.

Moreover, the liners described herein may be used in connection with the optical sensors described herein.

Again, the foregoing examples of manners of combining the aspects of the present disclosure are non-limiting.

Having thus described several aspects and embodiments of the technology of this application, it is to be appreciated that various alterations, modifications, and improvements will readily occur to those of ordinary skill in the art. Such alterations, modifications, and improvements are intended to be within the spirit and scope of the technology described in the application. For example, those of ordinary skill in the art will readily envision a variety of other means and/or structures for performing the function and/or obtaining the results and/or one or more of the advantages described herein, and each of such variations and/or modifications is deemed to be within the scope of the embodiments described herein. Those skilled in the art will recognize, or be able to ascertain using no more than routine experimentation, many equivalents to the specific embodiments described herein. It is, therefore, to be understood that the foregoing embodiments are presented by way of example only and that, within the scope of the appended claims and equivalents thereto, inventive embodiments may be practiced otherwise than as specifically described. In addition, any combination of two or more features, systems, articles, materials, kits, and/or methods described herein, if such features, systems, articles, materials, kits, and/or methods are not mutually inconsistent, is included within the scope of the present disclosure.

The above-described embodiments can be implemented in any of numerous ways. One or more aspects and embodiments of the present application involving the performance of processes or methods may utilize program instructions executable by a device (e.g., a computer, a processor, or other device) to perform, or control performance of, the processes or methods. In this respect, various inventive concepts may be embodied as a computer readable storage medium (or multiple computer readable storage media) (e.g., a computer memory, one or more floppy discs, compact discs, optical discs, magnetic tapes, flash memories, circuit configurations in Field Programmable Gate Arrays or other semiconductor devices, or other tangible computer storage medium) encoded with one or more programs that, when executed on one or more computers or other processors, perform methods that implement one or more of the various embodiments described above. The computer readable medium or media can be transportable, such that the program or programs stored thereon can be loaded onto one or more different computers or other processors to implement various ones of the aspects described above. In some embodiments, computer readable media may be non-transitory media.

The terms “program” or “software” are used herein in a generic sense to refer to any type of computer code or set of computer-executable instructions that can be employed to program a computer or other processor to implement various aspects as described above. Additionally, it should be appreciated that according to one aspect, one or more computer programs that when executed perform methods of the present application need not reside on a single computer or processor, but may be distributed in a modular fashion among a number of different computers or processors to implement various aspects of the present application.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

Also, data structures may be stored in computer-readable media in any suitable form. For simplicity of illustration, data structures may be shown to have fields that are related through location in the data structure. Such relationships may likewise be achieved by assigning storage for the fields with locations in a computer-readable medium that convey relationship between the fields. However, any suitable mechanism may be used to establish a relationship between information in fields of a data structure, including through the use of pointers, tags or other mechanisms that establish relationship between data elements.

When implemented in software, the software code can be executed on any suitable processor or collection of processors, whether provided in a single computer or distributed among multiple computers.

Further, it should be appreciated that a computer may be embodied in any of a number of forms, such as a rack-mounted computer, a desktop computer, a laptop computer, or a tablet computer, as non-limiting examples. Additionally, a computer may be embedded in a device not generally regarded as a computer but with suitable processing capabilities, including a Personal Digital Assistant (PDA), a smart phone or any other suitable portable or fixed electronic device.

Also, a computer may have one or more input and output devices. These devices can be used, among other things, to present a user interface. Examples of output devices that can be used to provide a user interface include printers or display screens for visual presentation of output and speakers or other sound generating devices for audible presentation of output. Examples of input devices that can be used for a user interface include keyboards, and pointing devices, such as mice, touch pads, and digitizing tablets. As another example, a computer may receive input information through speech recognition or in other audible formats.

Such computers may be interconnected by one or more networks in any suitable form, including a local area network or a wide area network, such as an enterprise network, and intelligent network (IN) or the Internet. Such networks may be based on any suitable technology and may operate according to any suitable protocol and may include wireless networks or wired networks.

Also, as described, some aspects may be embodied as one or more methods. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Elements other than those specifically identified by the “and/or” clause may optionally be present, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

What is claimed is:

1. A support, comprising:
first and second segments, the first segment being configured to couple to a rear portion of a subject's head and the second segment being in the shape of an elongated strip and configured to wrap substantially about a front portion and side portions of the subject's head, wherein the first segment and second segment each include at least one coupler configured to detachably couple an optical sensor to an inner surface of the first and second segments, respectively;
at least one fastener configured to detachably couple the first and second segments substantially in a loop such that the inner surface of the first segment and the inner surface of the second segment are directed inwardly toward a center of the loop;
a first tensioner anchored on the second segment and configured to adjust a sizing of the loop and comprising at least one first strap; and
a second tensioner anchored on the second segment and configured to adjust the sizing of the loop and comprising at least one second strap.

2. The support of claim 1, wherein the support is configured to mount to a subject's head when the at least one fastener couples the first and second segments substantially in a loop such that a top side of the subject's head is not obstructed by the support.

3. The support of claim 1, wherein the support is configured to mount to a subject's head when the at least one fastener couples the first and second segments substantially in a loop such that the support does not obstruct a drain positioned in the subject's head.

4. A support, comprising:
first and second segments, the first segment being configured to couple to a rear portion of a subject's head and the second segment being configured to couple to a front portion and side portions of the subject's head;
at least one fastener configured to couple the first and second segments substantially in a loop; and
at least one tensioner configured to adjust a sizing of the loop.

5. The support of claim 4, further comprising at least one coupler configured to mechanically couple to an optical sensor.

6. The support of claim 5, wherein the at least one coupler comprises an elastic strap configured to wrap around at least a portion of the optical sensor.

7. The support of claim 5, wherein the optical sensor comprises a plurality of corners, and wherein the at least one coupler comprises a plurality of elastic straps configured to secure the plurality of corners.

8. The support of claim 5, wherein the at least one coupler comprises a pouch.

9. The support of claim 5, wherein the at least one coupler is positioned on an inner portion of the support configured to be disposed proximate the subject's head.

10. The support of claim 5, wherein a surface of the support is configured to contact the optical sensor when the optical sensor is mechanically coupled to the support by the at least one coupler, and wherein at least a portion of the surface is configured to restrict movement of the optical sensor relative to the support when the optical sensor is mechanically coupled to the support.

11. The support of claim 4, further comprising at least one indicator to align placement of the support with a target location of the subject's head.

12. The support of claim 4, wherein at least one of the first segment and/or the second segment is formed at least in part of foam.

13. The support of claim 4, wherein the first segment and/or the second segment is flexible.

14. The support of claim 13, wherein the first segment and/or the second segment is flexible in at least two orthogonal directions.

15. The support of claim 13, wherein the first segment and/or the second segment is formed of a deformable material.

16. The support of claim 4, wherein the support is configured to conform, at least in part, to the subject's head.

17. The support of claim 4, wherein the first segment is configured to engage an occiput of the subject's head.

18. The support of claim 4, wherein the support is disposable.

19. An apparatus, comprising:
the support of claim 4; and
a liner configured to detachably engage with an optical sensor such that the liner is disposed between at least a portion of the optical sensor and the subject when the optical sensor is positioned to irradiate the subject with an optical signal and/or detect an optical signal from the subject.

20. An apparatus, comprising:
the support of claim 4; and
a substrate having a plurality of holes formed therein, the plurality of holes configured to align with a plurality of optical components of an optical sensor when the substrate is engaged with the optical sensor.
